(19) United States
(12) Patent Application Publication
(10) Pub. No.: US 2025/0368603 A1
(43) Pub. Date: Dec. 4, 2025

(54) PROCESS OF MAKING APOPTOSIS-INDUCING AGENTS

(71) Applicant: AbbVie Inc., North Chicago, IL (US)

(72) Inventors: Yi-Yin Ku, Buffalo Grove, IL (US); Timothy Grieme, Lake Bluff, IL (US); Kaid Harper, Mundelein, IL (US); Ji-An Jin, Gurnee, IL (US); Daniel Mack, Chicago, IL (US); James Marek, Buffalo Grove, IL (US); Eric Moschetta, Chicago, IL (US); Ketan Patel, Wheeling, IL (US); Yu-Ming Pu, Las Vegas, NV (US); Eric Sacia, Libertyville, IL (US); Timothy Towne, Grayslake, IL (US)

(21) Appl. No.: 18/686,409

(22) PCT Filed: Aug. 25, 2022
(86) PCT No.: PCT/US2022/075459

§ 371 Date: Feb. 24, 2024

(51) Int. Cl. C07C 319/14; B01J 35/39; C07C 315/02; C07D 295/13

(52) U.S. Cl. CPC C07C 319/14; B01J 35/39; C07C 315/02; C07D 295/13; C07C 2531/22

(57) ABSTRACT

Provided herein is a process for the preparation of an apoptosis-inducing agent, and chemical intermediates thereof. Also provided herein are novel chemical intermediates related to the process provided herein.

### FIELD OF THE DISCLOSURE

Provided herein are processes for the preparation of an apoptosis-inducing agent, and chemical intermediates thereof. Also provided herein are novel chemical intermediates related to the processes provided herein.

### CROSS-REFERENCE TO RELATED APPLICATIONS

This application claims the benefit of U.S. Provisional Patent Application Ser. No. 63/236,894, filed Aug. 25, 2021, the disclosures of which are incorporated herein by reference in their entirety.

### BACKGROUND OF THE DISCLOSURE

Proteins of the Bcl-2 family, e.g. Bcl-2, Bcl-xL and Mcl-1, enable cells to evade apoptosis. These proteins are implicated in cancer and other proliferative diseases. They are often upregulated in cancer cells, where they sequester and neutralize proapoptotic proteins, thus enabling the survival of the cancer cells despite the presence of apoptosis-triggering signals. Consequently, inhibitors of Bcl-2 family proteins are useful candidates for cancer therapy. Several inhibitors have been described, for example, in U.S. Pat. No. 7,390,799 B2.

A Bcl-2 family inhibitor is 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (ABT-263), the preparation of which is described in U.S. Pat. No. 7,390,799 B2 and in U.S. Pat. No. 8,168,784 B2. The molecular structure of ABT-263 is depicted below:

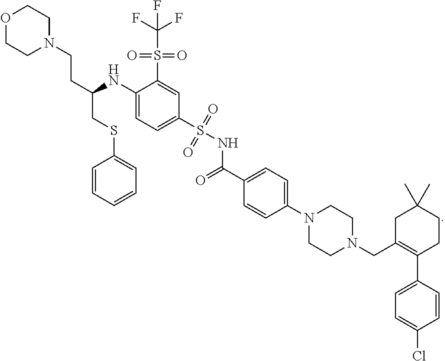 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

The synthesis of ABT-263 is disclosed in U.S. Pat. No. 7,390,799 B2 and in U.S. Pat. No. 8,168,784 B2. Use of the term “4-(4-{[2-(4-chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]benzamide” or “N-(4-(4-((2-(4-chlorophenyl)-5,5-dimethyl-1-cyclohex-1-en-1-yl)methyl)piperazine-1-yl)benzoyl)-4-(((1R)-3-(morpholin-4-yl)-1-((phenylsulfanyl)methyl)propyl)amino)-3-((trifluoromethyl)sulfonyl)benzenesulfonamide” are synonymous with navitoclax. However, a need remains for a scalable process that enable the efficient and cost-effective manufacture of ABT-263 on a commercial scale and in commercially useful quantities.

### SUMMARY OF THE DISCLOSURE

Provided herein are methods for the preparation of ABT-263.

In some aspects, methods are provided for the synthesis of 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide having structure (3-8):

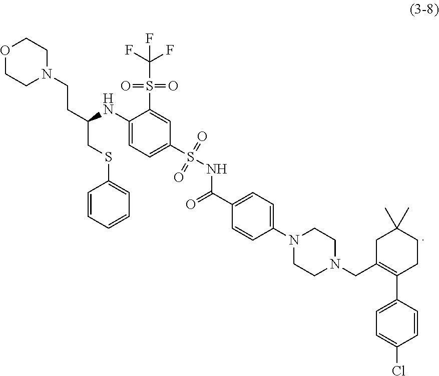 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some aspects, methods are provided for the synthesis of intermediates useful in the synthesis of 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide having structure (3-8).

In some embodiments, the present disclosure is directed to a method for preparing 1-chloro-2-[(trifluoromethyl)sulfanyl]benzene having structure (1-2):

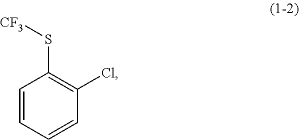 [SMILES: FC(F)(F)Sc1ccccc1Cl]

the method comprising alkylating 2-chlorobenzene-1-thiol having structure (1-1):

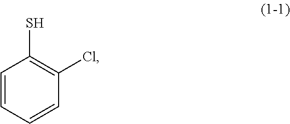

with trifluoroiodomethane (CF₃I) to thereby prepare 1-chloro-2-[(trifluoromethyl)sulfanyl]benzene. In some embodiments, alkylating 2-chlorobenzene-1-thiol is catalyzed by irradiation. In some embodiments, the mixture is irradiated with visible light. In some embodiments, alkylating 2-chlorobenzene-1-thiol is catalyzed by irradiation in the presence of a photoredox catalyst. In some embodiments, the photoredox catalyst is tris(2,2′-bipyridyl)dichlororuthenium(II) hexahydrate (Ru(bpy)₃Cl₂(H₂O)₆). In some embodiments, alkylating 2-chlorobenzene-1-thiol occurs in a reaction mixture prepared by combining a first feed solution comprising 2-chlorobenzene-1-thiol and a second feed solution comprising trifluoroiodomethane. In some embodiments, the first feed solution further comprises a photoredox catalyst. In some embodiments, the second feed solution further comprises an amine base. In some embodiments, the reaction mixture is irradiated with visible light.

In some embodiments, the present disclosure is directed to a method for preparing 1-chloro-2-(trifluoromethylsulfonyl)benzene having structure (1-3):

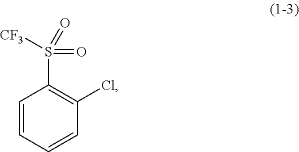 [SMILES: O=S(=O)(c1ccccc1Cl)C(F)(F)F]

the method comprising contacting 1-chloro-2-[(trifluoromethyl)sulfanyl]benzene with an oxidizing agent to thereby prepare 1-chloro-2-(trifluoromethylsulfonyl)benzene. In some embodiments, 1-chloro-2-[(trifluoromethyl)sulfanyl]benzene is prepared by alkylating 2-chlorobenzene-1-thiol with trifluoroiodomethane to thereby prepare 1-chloro-2-[(trifluoromethyl)sulfanyl]benzene. In some embodiments, the method is continuous from the preparation of 1-chloro-2-[(trifluoromethyl)sulfanyl]benzene from 2-chlorobenzene-1-thiol, and the preparation of 1-chloro-2-((trifluoromethyl)sulfonyl)benzene from 1-chloro-2-[(trifluoromethyl)sulfanyl]benzene.

In some embodiments, the present disclosure is directed to a method for preparing (2R)-4-(morpholin-4-yl)-1-(phenylsulfanyl)butan-2-amine L-tartrate having structure (2-6):

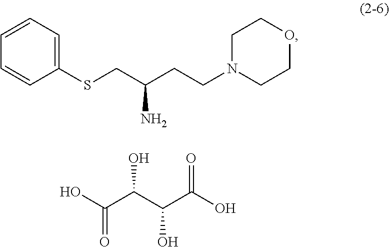

the method comprising contacting (3R)-3-amino-1-(morpholin-4-yl)-4-(phenylsulfanyl)butan-1-one having structure (2-5):

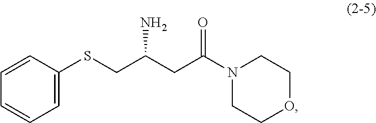 [SMILES: N[C@@H](CSc1ccccc1)CC(=O)N1CCOCC1]

with a borohydride and acid to thereby prepare (2R)-4-(morpholin-4-yl)-1-(phenylsulfanyl)butan-2-amine; contacting (2R)-4-(morpholin-4-yl)-1-(phenylsulfanyl)butan-2-amine with L-tartaric acid to thereby prepare (2R)-4-(morpholin-4-yl)-1-(phenylsulfanyl)butan-2-amine L-tartrate.

In some embodiments, the present disclosure is directed to a method for preparing 4-{[(2R)-4-(morpholin-4-yl)-1-(phenylsulfanyl)butan-2-yl]amino}-3-(trifluoromethanesulfonyl)benzene-1-sulfonamide having structure (2-7):

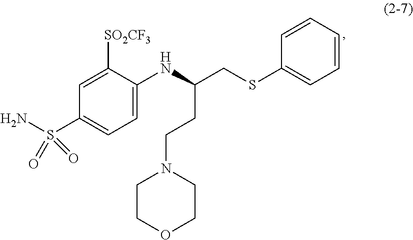

the method comprising converting (2R)-4-(morpholin-4-yl)-1-(phenylsulfanyl)butan-2-amine L-tartrate into free base (2R)-4-(morpholin-4-yl)-1-(phenylsulfanyl)butan-2-amine; and coupling free base (2R)-4-(morpholin-4-yl)-1-(phenylsulfanyl)butan-2-amine with 4-chloro-3-(trifluoromethanesulfonyl)benzene-1-sulfonamide having structure (1-5):

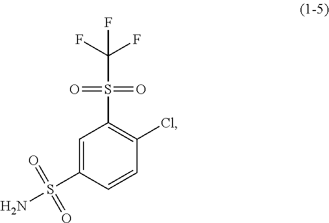 [SMILES: NS(=O)(=O)c1ccc(Cl)c(S(=O)(=O)C(F)(F)F)c1]

in an aprotic solvent catalyzed by a base to thereby prepare 4-{[(2R)-4-(morpholin-4-yl)-1-(phenylsulfanyl)butan-2-yl]amino}-3-(trifluoromethanesulfonyl)benzene-1-sulfonamide

In some embodiments, the present disclosure is directed to a method for preparing ethyl 4-{4-[(4′-chloro-4,4-dimethyl-3,4,5,6-tetrahydro[1,1′-biphenyl]-2-yl)methyl]piperazin-1-yl}benzoate having structure (3-6):

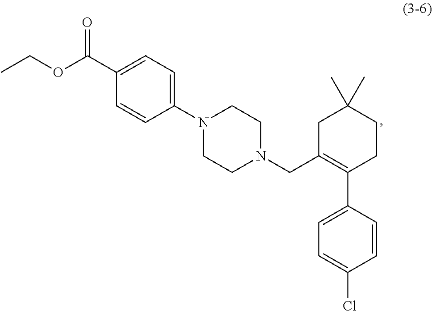 [SMILES: CCOC(=O)c1ccc(N2CCN(CC3=C(c4ccc(Cl)cc4)CCC(C)(C)C3)CC2)cc1]

the method comprising reacting 1-[(4′-chloro-4,4-dimethyl-3,4,5,6-tetrahydro[1,1′-biphenyl]-2-yl)methyl]piperazine having structure (3-5):

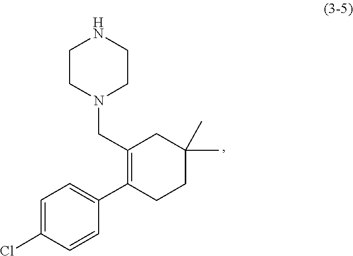

with ethyl 4-fluorobenzoate in the presence of an amine base in an aprotic solvent to thereby prepare ethyl 4-{4-[(4′-chloro-4,4-dimethyl-3,4,5,6-tetrahydro[1,1′-biphenyl]-2-yl)methyl]piperazin-1-yl}benzoate.

In some embodiments, the present disclosure is directed to a method for preparing navitoclax. The method comprising coupling 4-{4-[(4′-chloro-4,4-dimethyl-3,4,5,6-tetrahydro[1,1′-biphenyl]-2-yl)methyl]piperazin-1-yl}benzoic acid having structure (3-7):

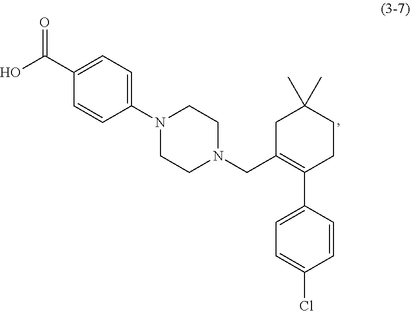 [SMILES: CC1(C)CCC(c2ccc(Cl)cc2)=C(CN2CCN(c3ccc(C(=O)O)cc3)CC2)C1]

with 4-{[(2R)-4-(morpholin-4-yl)-1-(phenylsulfanyl)butan-2-yl]amino}-3-(trifluoromethanesulfonyl)benzene-1-sulfonamide to thereby prepare 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (navitoclax); wherein 4-{[(2R)-4-(morpholin-4-yl)-1-(phenylsulfanyl)butan-2-yl]amino}-3-(trifluoromethanesulfonyl)benzene-1-sulfonamide is prepared by coupling a free base of (2R)-4-(morpholin-4-yl)-1-(phenylsulfanyl)butan-2-amine with 4-chloro-3-(trifluoromethanesulfonyl)benzene-1-sulfonamide in an aprotic solvent catalyzed by a base. In some embodiments, 4-{4-[(4′-chloro-4,4-dimethyl-3,4,5,6-tetrahydro[1,1′-biphenyl]-2-yl)methyl]piperazin-1-yl}benzoic acid is prepared by hydrolyzing ethyl 4-{4-[(4′-chloro-4,4-dimethyl-3,4,5,6-tetrahydro[1,1′-biphenyl]-2-yl)methyl]piperazin-1-yl}benzoate with a hydroxide base in a solvent composition comprising ethanol and water. In some embodiments, 4-chloro-3-(trifluoromethanesulfonyl)benzene-1-sulfonamide having structure (1-5) is prepared by reacting 4-chloro-3-(trifluoromethanesulfonyl)benzene-1-sulfonyl chloride with aqueous ammonia. In some embodiments, 4-chloro-3-(trifluoromethanesulfonyl)benzene-1-sulfonyl chloride is prepared by: (a) irradiating a mixture comprising 2-chlorobenzene-1-thiol a photoredox catalyst, and trifluoroiodomethane to thereby prepare 1-chloro-2-[(trifluoromethyl)sulfanyl]benzene (b) contacting 1-chloro-2-[(trifluoromethyl)sulfanyl]benzene with an oxidizing agent to thereby prepare 1-chloro-2-(trifluoromethylsulfonyl)benzene and, (c) reacting 1-chloro-2-(trifluoromethanesulfonyl)benzene with chlorosulfonic acid and then with thionyl chloride to thereby prepare 4-chloro-3-(trifluoromethanesulfonyl)benzene-1-sulfonyl chloride. In some embodiments, the method is continuous from the preparation of 1-chloro-2-[(trifluoromethyl)sulfanyl]benzene from 2-chlorobenzene-1-thiol of step (a), and from the preparation of 1-chloro-2-(trifluoromethylsulfonyl)benzene from 1-chloro-2-[(trifluoromethyl)sulfanyl]benzene of step (b).

In some embodiments, the present disclosure is directed to a compound having structure (1-4).

In some embodiments, the present disclosure is directed to a compound having structure (1-5).

### BRIEF DESCRIPTION OF THE DRAWINGS

FIG. 1. depicts a schematic for the laser continuous stirred tank reactor (CSTR) flow conditions for Example 1.

FIG. 2. depicts a schematic for the continuous washing workup under flow reaction conditions of the trifluoromethylation reaction for Example 1.

FIG. 3 depicts a schematic of the flow oxidation conditions of Example 2.

FIGS. 4A, 4B, and 4C depict the flow schematics for the reactions of Example 1 and Example 3.

FIGS. 5A, 5B, and 5C depict the flow schematics for the reactions of Example 2 and Example 3.

FIG. 6 depicts the schematics for the plug flow photo reaction of Example 4.

### DETAILED DESCRIPTION OF THE DISCLOSURE

The present disclosure is directed to commercially scalable, synthetic processes for making navitoclax active pharmaceutical ingredient (API), and novel intermediates used in the processes. The navitoclax API derived from the processes described herein is suitable for inclusion in commercial drug product.

### 1. Synthetic Processes for Making Navitoclax

Provided herein is a method for the preparation of 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (ABT-263) having structure (3-8):

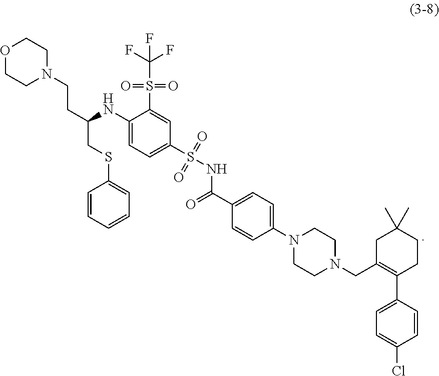 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

The present disclosure is additionally directed to methods of preparing intermediates useful in the preparation of ABT-263. Specifically, the present disclosure is directed to methods of preparing intermediates 4-{[(2R)-4-(morpholin-4-yl)-1-(phenylsulfanyl)butan-2-yl]amino}-3-(trifluoromethanesulfonyl)benzene-1-sulfonamide having structure (2-7) and 4-{4-[(4′-chloro-4,4-dimethyl-3,4,5,6-tetrahydro[1,1′-biphenyl]-2-yl)methyl]piperazin-1-yl}benzoic acid (3-7), which are coupled to prepare ABT-263:

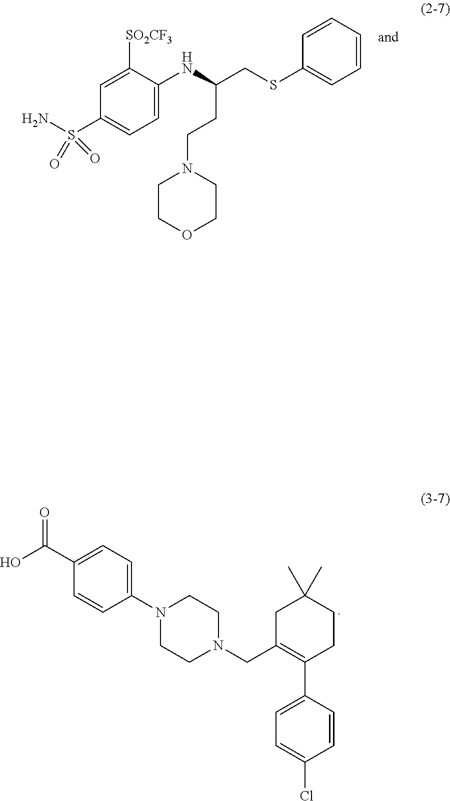

Intermediate 4-{[(2R)-4-(morpholin-4-yl)-1-(phenylsulfanyl)butan-2-yl]amino}-3-(trifluoromethanesulfonyl)benzene-1-sulfonamide having structure (2-7) is prepared by coupling intermediates 4-chloro-3-(trifluoromethanesulfonyl)benzene-1-sulfonamide (1-5) and (2R)-4-(morpholin-4-yl)-1-(phenylsulfanyl)butan-2-amine L-tartrate (2-6).

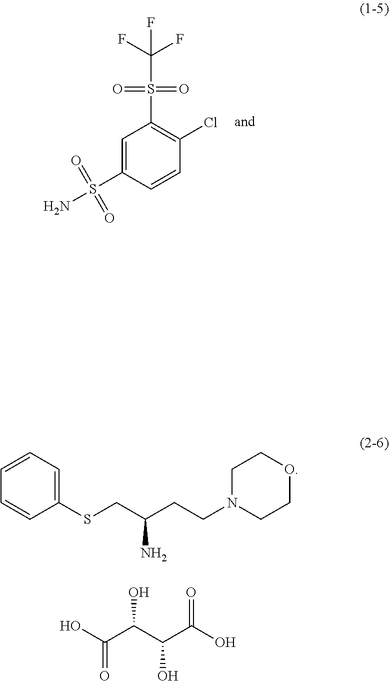

### Synthesis of 4-chloro-3-(trifluoromethanesulfonyl)benzene-1-sulfonamide (1-5)

According to the present disclosure, the starting point for the synthesis of 4-chloro-3-(trifluoromethanesulfonyl)benzene-1-sulfonamide having structure (1-5) is 2-chlorobenzene-1-thiol having structure (1-1):

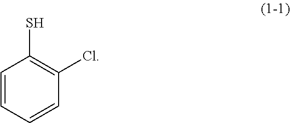

2-Chlorobenzene-1-thiol having structure (1-1) is alkylated with a trifluoromethylation agent to thereby prepare 1-chloro-2-[(trifluoromethyl)sulfanyl]benzene having structure (1-2):

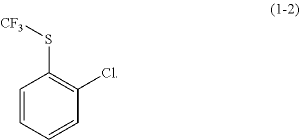 [SMILES: FC(F)(F)Sc1ccccc1Cl]

2-Chlorobenzene-1-thiol having structure (1-1) is alkylated with a trifluoroiodomethane to thereby prepare 1-chloro-2-[(trifluoromethyl)sulfanyl]benzene having structure (1-2):

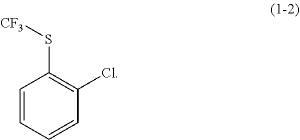 [SMILES: FC(F)(F)Sc1ccccc1Cl]

The trifluoromethylation of 2-chlorobenzene-1-thiol (1-1) occurs with a trifluoromethylation agent in the presence of a base. Exemplary trifluoromethylation agents include trifluoroiodomethane (CF₃I), trifluoromethyltrimethylsilane (CF₃SiMe₃), trifluoromethane (CF₃H), and trifluoromethanesulfonyl chloride (CF₃SO₂Cl). In some embodiments, the alkylation with a trifluoromethylation agent occurs with amine base, preferably a tertiary amine. Exemplary tertiary amine include trimethylamine, triethylamine (TEA), N,N-diisopropylethylamine, N,N-dimethylpyridin-4-amine, 1,1,3,3-tetramethylguanidine (TMG), 2-tert-butyl-1,1,3,3-tetramethylguanidine, 1,5,7-triazabicyclo[4.4.0]dec-5-ene, 7-methyl-1,5,7-triazabicyclo[4.4.0]dec-5-ene, or 1,8-diazabicyclo[5.4.0]undec-7-ene.

The reaction may occur in a batch process or flow reaction conditions. In some embodiments, the reaction may be catalyzed by irradiation. A reaction catalyzed by irradiation may occur with or without a photoredox catalyst. Preferably, the reaction mixture includes a photoredox catalyst, which ensures consistent results. The photoredox flow reaction preferably occurs in an aprotic solvent, such as acetonitrile. Examples of photoredox catalysts useful in the synthesis of compounds in this disclosure include, but are not limited to, tris(2,2′-bipyridyl)dichlororuthenium(II) hexahydrate (Ru(bpy)₃Cl₂(H₂O)₆), (2,2′-bipyridine)bis(2-phenylpyridinato)iridium(III) hexafluorophosphate, and tris[5-fluoro-2-(2-pyridinyl-kN)phenyl-kC]iridium(III) Ir(p-F-ppy)₃.

In some embodiments, the trifluoromethylation agent is trifluoroiodomethane (CF₃I) and the tertiary amine is 1,1,3,3-tetramethylguanidine (TMG). According to some embodiments, combining the photoredox catalyst and 2-chlorobenzene-1-thiol (1-1) in one feed, and TMG and CF₃I in another feed, met the criteria of being stable over a sufficiently long period of time, gave consistent reaction performance, and/or was amenable to the available processing equipment. This is important because CF₃I is a gas at ambient pressure and temperature and poses unique challenges. It can be dissolved in solvents, but over the typical processing time of 100 hours, evaporation of CF₃I will be observed. However, CF₃I does form a complex in solution with TMG. At −5° C. to 0° C., the complex is stable throughout the reaction processing time. For example, 2-chlorobenzene-1-thiol (1-1) and photocatalyst solution demonstrate stability in excess of 14 days. Due to the low boiling point of CF₃I (−20° C.), loss of potency of the solution during the projected processing time (e.g., approximately 100 hours) was also a concern. TMG was therefore selected due to the bench stable complex it forms with CF₃I. Further, TMG can be readily removed during the workup via acidic washes. The TMG-CF₃I solution is preferably held at or below temperatures of −5° C. to 0° C. to ensure adequate stability. The TMG-CF₃I is preferably at a molar excess of 2-chlorobenzene-1-thiol (1-1) to enable more complete trifluoromethylation, such as at least a 1.1:1 molar ratio of TMG-CF₃I:2-chlorobenzene-1-thiol (1-1), at least a 1.2:1 molar ratio, or more.

In some embodiments, the reaction occurs under photoredox condition catalyzed by irradiation. In some embodiments, the trifluoromethylation reaction of 2-chlorobenzene-1-thiol (1-1) to prepare 1-chloro-2-[(trifluoromethyl)sulfanyl]benzene (1-2) is mediated by visible light, which encompasses about 450 nm to about 700 nm wavelengths, preferably between about 405 nm and about 530 nm, and more preferably about 450 nm. The visible light source may be a visible light laser, e.g., a fiber-coupled laser, which provides the advantage of segregation of the heat associated with light generation from the reactor itself allowing for greater temperature control. Suitable lasers are those with an output wavelength within the above recited wavelength ranges and suitable power for the continuous flow reaction. The visible light source may alternatively be a light emitting diode (LED). The output power is preferably at least 10 W, such as at least 20 W, at least 25 W, or at least 30 W. Suitable surface irradiation on the reaction composition may vary between about 10 mW/cm² to about 30 mW/cm², and such as between about 15 mW/cm².

The reaction may be characterized as a photoredox flow reaction since the reaction mixture is formed by the combination of two solutions, which is passed through a photoreactor chamber equipped with visible light, or is pumped into a reaction tank, which is irradiated in order to catalyze the trifluoromethylation of 2-chlorobenzene-1-thiol (1-1) to prepare 1-chloro-2-[(trifluoromethyl)sulfanyl]benzene (1-2). According to some embodiments, a first feed solution is prepared comprising 2-chlorobenzene-1-thiol and the photoredox catalyst, and a second feed solution is prepared comprising the amine base, preferably a tertiary amine, and the trifluoromethylation agent. The feed solutions are preferably prepared in an aprotic solvent, preferably the same aprotic solvent for each, such as acetonitrile. Preferably the feed solutions are cooled to or below a temperature of about 0° C. or even −5° C. prior to combining in a reactor. The feed solutions are then fed into a reactor, e.g., a holding tank, which is irradiated. Preferably, the reactor comprises aprotic solvent, such as acetonitrile, and the reactor may be optionally charged with photoredox catalyst. The holding tank is agitated, e.g., by a stir bar. As the feed solutions enter the holding tank, they combine to form a mixture that is irradiated, which catalyzes the trifluoromethylation of 2-chlorobenzene-1-thiol (1-1). The residence time in the reactor may be relatively short, such as on the order of about 30 seconds to about 30 minutes, or between about 1 minute and about 15 minutes. The reaction is continuous, making it suitable for preparing commercial quantities of ABT-263. A flow reaction enables commercial scale-up by reacting only a portion of the reaction at a time, and therefore, the volumes irradiated by the light source are lower. The light source may efficiently irradiate the entire reaction vessel. Scale up of a batch process is far more difficult since the light source may not efficiently irradiate a large volume reaction vessel. The product 1-chloro-2-[(trifluoromethyl)sulfanyl]benzene (1-2) is collected in a collection vessel. The entire reaction is a flow reaction since feed solution is continually fed into the reaction vessel, and once a residence time has been established, product solution is continually collected in the collection vessel. The reaction proceeds until the feed solutions are exhausted. The product 1-chloro-2-[(trifluoromethyl)sulfanyl]benzene (1-2) may be optionally isolated from the solvent and purified. However, the crude 1-chloro-2-[(trifluoromethyl)sulfanyl]benzene (1-2) may alternatively be washed and then proceed directly to an oxidation reactor to prepare 1-chloro-2-(trifluoromethanesulfonyl)benzene (1-3) having structure:

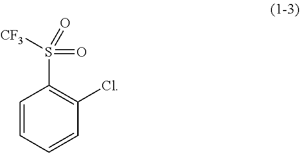 [SMILES: O=S(=O)(c1ccccc1Cl)C(F)(F)F]

The oxidation of 1-chloro-2-[(trifluoromethyl)sulfanyl]benzene (1-2) to prepare 1-chloro-2-(trifluoromethanesulfonyl)benzene (1-3) may be performed by a batch process or is advantageously performed in a flow reaction. Crude 1-chloro-2-[(trifluoromethyl)sulfanyl]benzene (1-2) obtained from the photoredox flow reaction is combined with a composition comprising an oxidizing agent, such as NaIO₄ for the batch process or orthoperiodic acid (H₅IO₆) for a flow reaction, an aprotic solvent, and optionally an oxidation catalyst, such as RuCl₃. Examples of oxidizing agents include, but are not limited to, orthoperiodic acid (H₅IO₆), sodium periodate (NaIO₄), OXONE® (potassium peroxymonosulfate, KHSO₅), and sodium bromate. The oxidizing agent, e.g., orthoperiodic acid (H₅IO₆), is preferably at a molar excess of 1-chloro-2-[(trifluoromethyl)sulfanyl]benzene (1-2) to enable more complete oxidation; for example, in one embodiment at least a 2.1:1 molar ratio of H₅IO₆:1-chloro-2-[(trifluoromethyl)sulfanyl]benzene (1-2) may be used, such as a 2.2:1, a 2.3:1, or even a 2.4:1 molar ratio.

The oxidation of 1-chloro-2-[(trifluoromethyl)sulfanyl]benzene (1-2) to prepare 1-chloro-2-(trifluoromethanesulfonyl)benzene (1-3) also preferably occurs under flow conditions. The reactor, e.g., a holding tank, may be equipped with a plurality of inlets, such that the components of the reaction may be prepared in separate feed solutions and combined in the reactor. The holding tank may also be equipped with agitation, e.g., a stir bar or impellor, to ensure mixing of the feed solutions. In some embodiments, the feed solutions include crude 1-chloro-2-[(trifluoromethyl)sulfanyl]benzene (1-2) from the photoredox flow reaction, an oxidant solution such as orthoperiodic acid in an aqueous solution, and an aqueous catalyst solution. An optional aprotic solvent solution may also be fed into the reactor. As the feed solutions are mixed in the reactor, 1-chloro-2-[(trifluoromethyl)sulfanyl]benzene (1-2) is oxidized to prepare product 1-chloro-2-(trifluoromethanesulfonyl)benzene (1-3). The reactor may be equipped with an outlet and collection vessel to collect the product.

The above described flow methods enable the “continuous” preparation of 1-chloro-2-(trifluoromethanesulfonyl)benzene (1-3) from 2-chlorobenzene-1-thiol (1-1) starting material. “Continuous” in the context of the present disclosure means that the photoredox flow reaction and the flow reaction may be connected in a continuous flow as 2-chlorobenzene-1-thiol (1-1) is continuously trifluoromethylated to 1-chloro-2-[(trifluoromethyl)sulfanyl]benzene (1-2), which is continuously fed as a crude solution into the oxidation reactor to thereby prepare 1-chloro-2-(trifluoromethanesulfonyl)benzene (1-3). The continuous flow method enables each solution to be prepared separately with advantageous stability control, e.g., temperature, stirring, etc. Moreover, combining feed solutions enables rapid reaction with relatively short residence time in each reactor, thereby enhancing throughput. As demonstrated in the Examples, the reaction further proceeds to high yield.

According to the method of the present invention, 1-chloro-2-(trifluoromethanesulfonyl)benzene (1-3) is reacted with chlorosulfonic acid and then with thionyl chloride to thereby prepare 4-chloro-3-(trifluoromethanesulfonyl)benzene-1-sulfonyl chloride having structure (1-4):

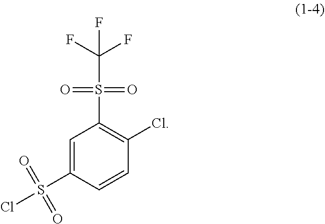 [SMILES: O=S(=O)(Cl)c1ccc(Cl)c(S(=O)(=O)C(F)(F)F)c1]

4-chloro-3-(trifluoromethanesulfonyl)benzene-1-sulfonyl chloride having structure (1-4) is reacted with aqueous ammonia to thereby prepare 4-chloro-3-(trifluoromethanesulfonyl)benzene-1-sulfonamide having structure (1-5):

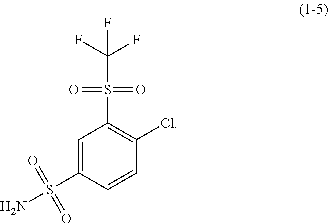 [SMILES: NS(=O)(=O)c1ccc(Cl)c(S(=O)(=O)C(F)(F)F)c1]

4-chloro-3-(trifluoromethanesulfonyl)benzene-1-sulfonamide having structure (1-5) is then coupled with (2R)-4-(morpholin-4-yl)-1-(phenylsulfanyl)butan-2-amine L-tartrate (2-6) to thereby prepare 4-{[(2R)-4-(morpholin-4-yl)-1-(phenylsulfanyl)butan-2-yl]amino}-3-(trifluoromethanesulfonyl)benzene-1-sulfonamide having structure (2-7).

### Synthesis of 4-{[(2R)-4-(morpholin-4-yl)-1-(phenylsulfanyl)butan-2-yl]amino}-3-(trifluoromethanesulfonyl)benzene-1-sulfonamide having structure (2-7)

According to the present disclosure, the starting point for the synthesis of 4-{[(2R)-4-(morpholin-4-yl)-1-(phenylsulfanyl)butan-2-yl]amino}-3-(trifluoromethanesulfonyl)benzene-1-sulfonamide having structure (2-7) is benzyl [(3R)-5-oxooxolan-3-yl]carbamate having structure (2-1):

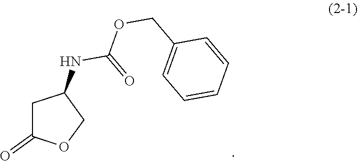 [SMILES: O=C1C[C@@H](NC(=O)OCc2ccccc2)CO1]

Benzyl [(3R)-5-oxooxolan-3-yl]carbamate (2-1) is reacted with morpholine an aprotic solvent to thereby prepare benzyl [(2R)-1-hydroxy-4-(morpholin-4-yl)-4-oxobutan-2-yl]carbamate having structure (2-2):

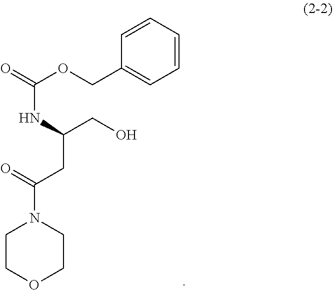 [SMILES: O=C(N[C@@H](CO)CC(=O)N1CCOCC1)OCc1ccccc1]

Benzyl [(2R)-1-hydroxy-4-(morpholin-4-yl)-4-oxobutan-2-yl]carbamate having structure (2-2) is reacted with tertiary amine and methanesulfonyl chloride in aprotic solvent to thereby prepare intermediate (2R)-2-{[(benzyloxy) carbonyl]amino}-4-(morpholin-4-yl)-4-oxobutyl methanesulfonate having structure (2-3):

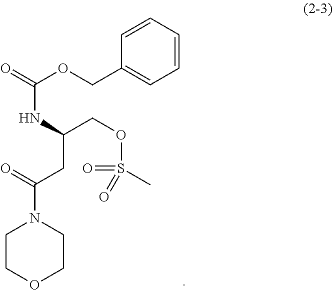 [SMILES: CS(=O)(=O)OC[C@@H](CC(=O)N1CCOCC1)NC(=O)OCc1ccccc1]

(2R)-2-{[(Benzyloxy) carbonyl]amino}-4-(morpholin-4-yl)-4-oxobutyl methanesulfonate having structure (2-3) undergoes further reaction with sodium benzenethiolate in aprotic solvent to thereby form benzyl [(2R)-4-(morpholin-4-yl)-4-oxo-1-(phenylsulfanyl)butan-2-yl]carbamate having structure (2-4):

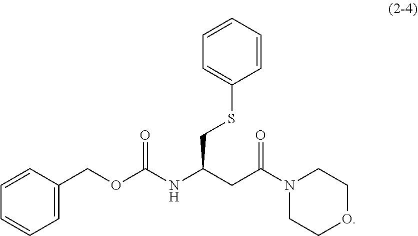 [SMILES: O=C(N[C@@H](CSc1ccccc1)CC(=O)N1CCOCC1)OCc1ccccc1]

Benzyl [(2R)-4-(morpholin-4-yl)-4-oxo-1-(phenylsulfanyl)butan-2-yl]carbamate having structure (2-4) is treated with acid to removing the protecting group, thereby yielding (3R)-3-amino-1-(morpholin-4-yl)-4-(phenylsulfanyl)butan-1-one having structure (2-5):

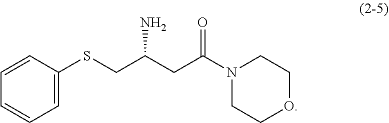 [SMILES: N[C@@H](CSc1ccccc1)CC(=O)N1CCOCC1]

(3R)-3-Amino-1-(morpholin-4-yl)-4-(phenylsulfanyl)butan-1-one having structure (2-5) is then reduced with sodium borohydride in the presence of acid to thereby form (2R)-4-(morpholin-4-yl)-1-(phenylsulfanyl)butan-2-amine L-tartrate having structure (2-6):

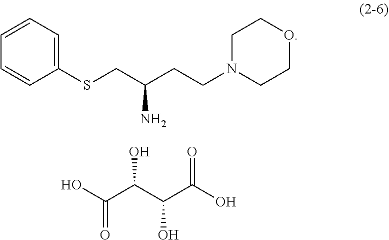

The reduction of (3R)-3-amino-1-(morpholin-4-yl)-4-(phenylsulfanyl)butan-1-one (2-5) preferably occurs in a cooled solution having a temperature of less than about 10° C., such as between about-5° C. and about 10° C., in an aprotic solvent, and preferably an anhydrous aprotic solvent, such a tetrahydrofuran. (3R)-3-Amino-1-(morpholin-4-yl)-4-(phenylsulfanyl)butan-1-one (2-5) is added to a solution of sodium borohydride in aprotic solvent, such as tetrahydrofuran. The reaction is catalyzed by acid. The organic phase including sodium borohydride and (3R)-3-amino-1-(morpholin-4-yl)-4-(phenylsulfanyl)butan-1-one (2-5) may be dosed with an acid, such as sulfuric acid, hydrochloric acid, or a combination thereof. The acid may be slowly dosed over several hours to ensure that the internal temperature of the solution remains below about 10° C., such as between about −5° C. and about 10° C. After the reaction, the (2R)-4-(morpholin-4-yl)-1-(phenylsulfanyl)butan-2-amine is contacted with L-tartaric acid in a protic solvent, such as a mixture of isopropyl acetate, methanol, and water, to thereby prepare (2R)-4-(morpholin-4-yl)-1-(phenylsulfanyl)butan-2-amine L-tartrate having structure of formula (2-6).

(2R)-4-(Morpholin-4-yl)-1-(phenylsulfanyl)butan-2-amine L-tartrate (2-6) prepared by the previous step is converted to the free base, which is then coupled with 4-chloro-3-(trifluoromethanesulfonyl)benzene-1-sulfonamide having structure (1-5) in an aprotic solvent catalyzed by a base to thereby prepare 4-{[(2R)-4-(morpholin-4-yl)-1-(phenylsulfanyl)butan-2-yl]amino}-3-(trifluoromethanesulfonyl)benzene-1-sulfonamide having structure (2-7):

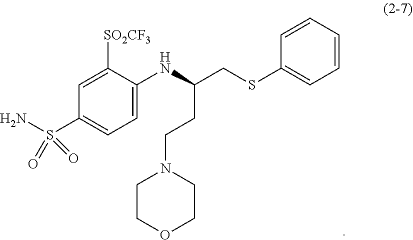

### Synthesis of 4-{4-[(4′-chloro-4,4-dimethyl-3,4,5,6-tetrahydro[1,1′-biphenyl]-2-yl)methyl]piperazin-1-yl}benzoic acid (3-7)

According to the present disclosure, the starting point for the synthesis of 4-{4-[(4′-chloro-4,4-dimethyl-3,4,5,6-tetrahydro[1,1′-biphenyl]-2-yl)methyl]piperazin-1-yl}benzoic acid (3-7) is 4,4-dimethylcyclohexan-1-one (3-1):

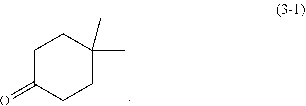

4,4-Dimethylcyclohexan-1-one (3-1) is reacted with phosphorus oxychloride and N,N-dimethylformamide in aprotic solvent such as dichloromethane to thereby prepare 2-chloro-5,5-dimethylcyclohex-1-ene-1-carbaldehyde having structure (3-2):

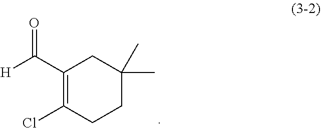

2-Chloro-5,5-dimethylcyclohex-1-ene-1-carbaldehyde (3-2) may be reacted with 4-chlorophenylboronic acid or other coupling partner under Suzuki reaction conditions to thereby prepare 4′-chloro-4,4-dimethyl-3,4,5,6-tetrahydro-[1,1′-biphenyl]-2-carbaldehyde having structure (3-3):

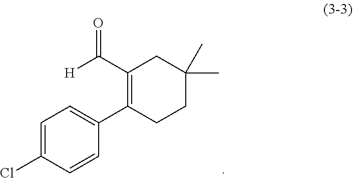 [SMILES: CC1(C)CCC(c2ccc(Cl)cc2)=C(C=O)C1]

4′-Chloro-4,4-dimethyl-3,4,5,6-tetrahydro-[1,1′-biphenyl]-2-carbaldehyde (3-3) may be reacted with tert-butyl piperazine-1-carboxylate under reductive amination conditions to thereby prepare tert-butyl 4-[(4′-chloro-4,4-dimethyl-3,4,5,6-tetrahydro[1,1′-biphenyl]-2-yl)methyl]piperazine-1-carboxylate having structure (3-4):

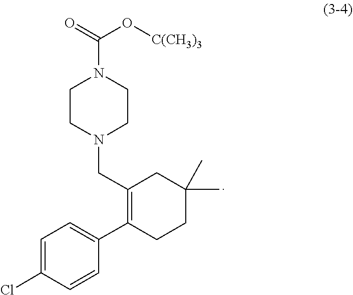

The protecting group is thereafter removed from tert-butyl 4-[(4′-chloro-4,4-dimethyl-3,4,5,6-tetrahydro[1,1′-biphenyl]-2-yl)methyl]piperazine-1-carboxylate (3-4) to thereby prepare 1-[(4′-chloro-4,4-dimethyl-3,4,5,6-tetrahydro[1,1′-biphenyl]-2-yl)methyl]piperazine having structure (3-5):

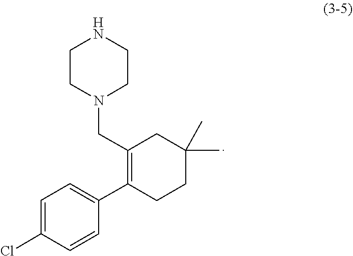

1-[(4′-Chloro-4,4-dimethyl-3,4,5,6-tetrahydro[1,1′-biphenyl]-2-yl)methyl]piperazine (3-5) is coupled with ethyl 4-fluorobenzoate in the presence of a base and in an aprotic solvent to thereby prepare ethyl 4-{4-[(4′-chloro-4,4-dimethyl-3,4,5,6-tetrahydro[1,1′-biphenyl]-2-yl)methyl]piperazin-1-yl}benzoate having structure (3-6):

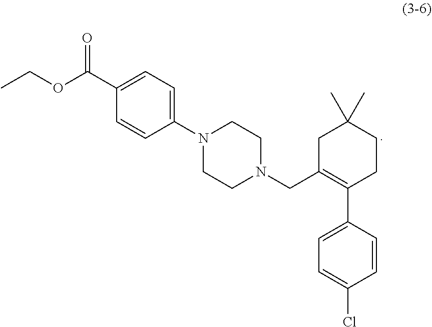 [SMILES: CCOC(=O)c1ccc(N2CCN(CC3=C(c4ccc(Cl)cc4)CCC(C)(C)C3)CC2)cc1]

Ethyl 4-{4-[(4′-chloro-4,4-dimethyl-3,4,5,6-tetrahydro[1,1′-biphenyl]-2-yl)methyl]piperazin-1-yl}benzoate (3-6) is saponified with a hydroxide base in an aqueous or protic solvent to thereby prepare 4-{4-[(4′-chloro-4,4-dimethyl-3,4,5,6-tetrahydro[1,1′-biphenyl]-2-yl)methyl]piperazin-1-yl}benzoic acid having structure (3-7):

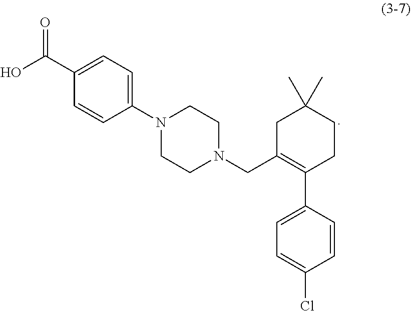 [SMILES: CC1(C)CCC(c2ccc(Cl)cc2)=C(CN2CCN(c3ccc(C(=O)O)cc3)CC2)C1]

4-{4-[(4′-Chloro-4,4-dimethyl-3,4,5,6-tetrahydro[1,1′-biphenyl]-2-yl)methyl]piperazin-1-yl}benzoic acid (3-7) is coupled with 4-{[(2R)-4-(morpholin-4-yl)-1-(phenylsulfanyl)butan-2-yl]amino}-3-(trifluoromethanesulfonyl)benzene-1-sulfonamide having structure (2-7) to thereby prepare 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (ABT-263) having structure (3-8):

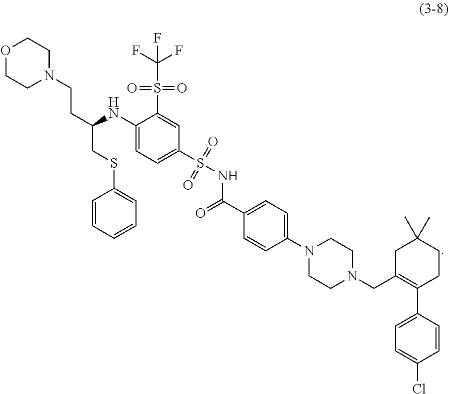 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Methods of Making Exemplary Compounds

The compounds of the present disclosure may be better understood in connection with the following synthetic schemes and methods which illustrate a means by which the compounds can be prepared. The compounds of the present disclosure can be prepared by a variety of synthetic procedures. Representative synthetic procedures are shown in, but not limited to, Schemes 1-3.

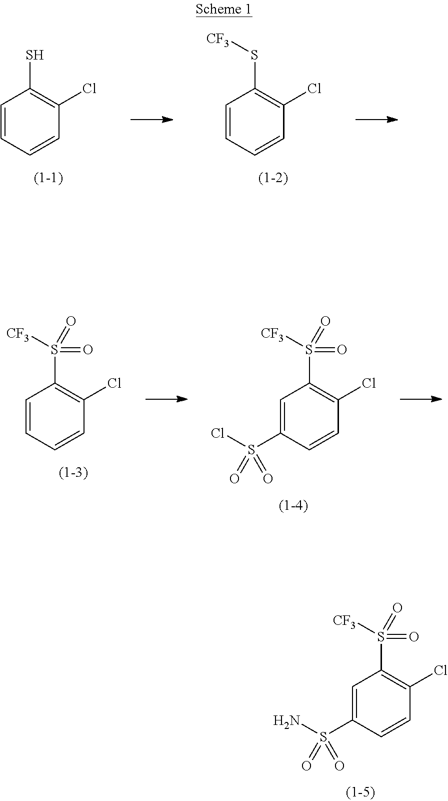 [SMILES: Sc1ccccc1Cl ; FC(F)(F)Sc1ccccc1Cl ; O=S(=O)(c1ccccc1Cl)C(F)(F)F ; O=S(=O)(Cl)c1ccc(Cl)c(S(=O)(=O)C(F)(F)F)c1 ; NS(=O)(=O)c1ccc(Cl)c(S(=O)(=O)C(F)(F)F)c1]

As shown in Scheme 1, the compound of formula (1-5) can be prepared from the compound of formula (1-1). The compound of formula (1-1), 2-chlorobenzene-1-thiol, can be alkylated with CF₃I under batch reaction conditions or under or under photoredox flow chemistry conditions to give the compound of formula (1-2). The compound of formula (1-2) can be oxidized with an oxidant such as but not limited to NaIO₄ or periodic acid catalyzed by ruthenium(III) chloride hydrate in an optionally warmed mixture of acetonitrile and water in a batch reactor or a flow reactor to give the compound of formula (1-3). The compound of formula (1-3) can be reacted first with chlorosulfonic acid at an elevated temperature and then with thionyl chloride to give the compound of formula (1-4). The compound of formula (1-4) can be reacted with aqueous ammonia in a solvent such as but not limited to chilled isopropyl acetate to give the compound of formula (1-5), 4-chloro-3-(trifluoromethanesulfonyl)benzene-1-sulfonamide

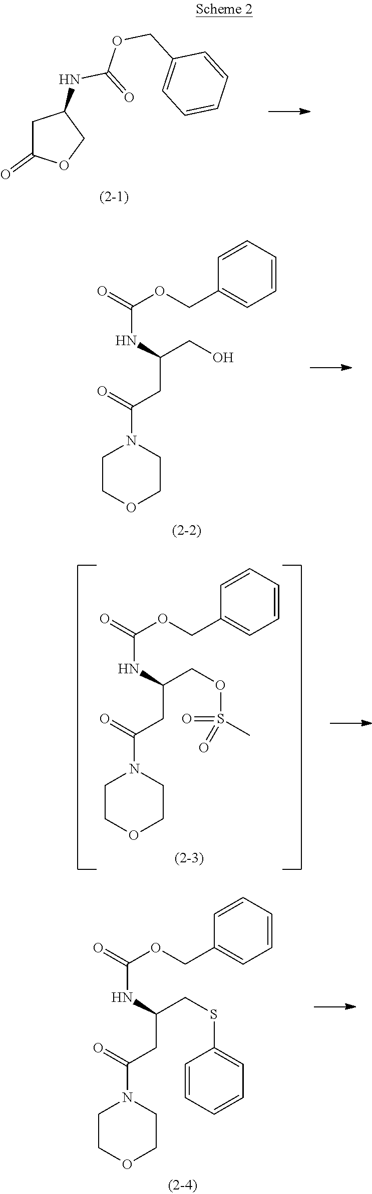

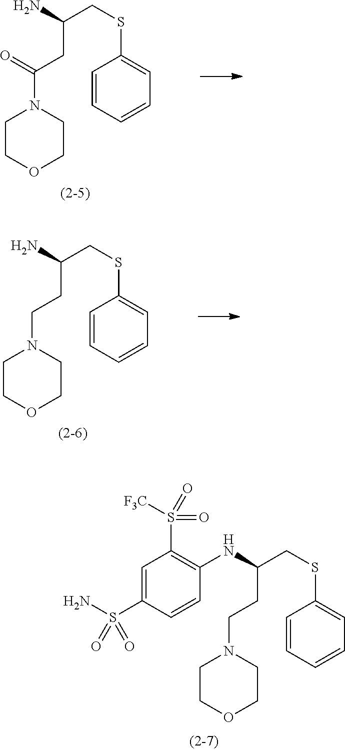 [SMILES: N[C@@H](CSc1ccccc1)CC(=O)N1CCOCC1 ; N[C@H](CCN1CCOCC1)CSc1ccccc1 ; NS(=O)(=O)c1ccc(N[C@H](CCN2CCOCC2)CSc2ccccc2)c(S(=O)(=O)C(F)(F)F)c1]

As shown in Scheme 2, the compound of formula (2-7) can be prepared from the compound of formula (2-1). The compound of formula (2-1), benzyl [(3R)-5-oxooxolan-3-yl]carbamate, can be reacted with morpholine in a solvent such as but not limited to heated 2-methyltetrahydrofuran to give the compound of formula (2-2). The compound of formula (2-2) can be reacted in a solvent such as but not limited to cooled acetonitrile with a base such as triethylamine and methanesulfonyl chloride to give the intermediate of formula (2-3). The intermediate of formula (2-3) can be reacted with sodium benzenethiolate in a solvent such as cooled acetonitrile to give the compound of formula (2-4). The compound of formula (2-4) can be deprotected by treatment with 30% hydrobromic acid in acetic acid in a solvent mixture such as toluene and acetic acid at or near ambient temperature to give the compound of formula (2-5). The compound of formula (2-5) can be converted to the amine of formula (2-6) by treatment with sodium borohydride in a cooled solvent such as tetrahydrofuran. Following workup, the amine of formula (2-6) can be isolated as the L-tartrate salt from a mixture of isopropyl acetate, methanol, and water. The compound of formula (2-6) can be converted to the free base and then reacted with the compound of formula (1-5) in a solvent such as heated acetonitrile in the presence of a base such as 1,4-diazabicyclo[2.2.2]octane to give the compound of formula (2-7).

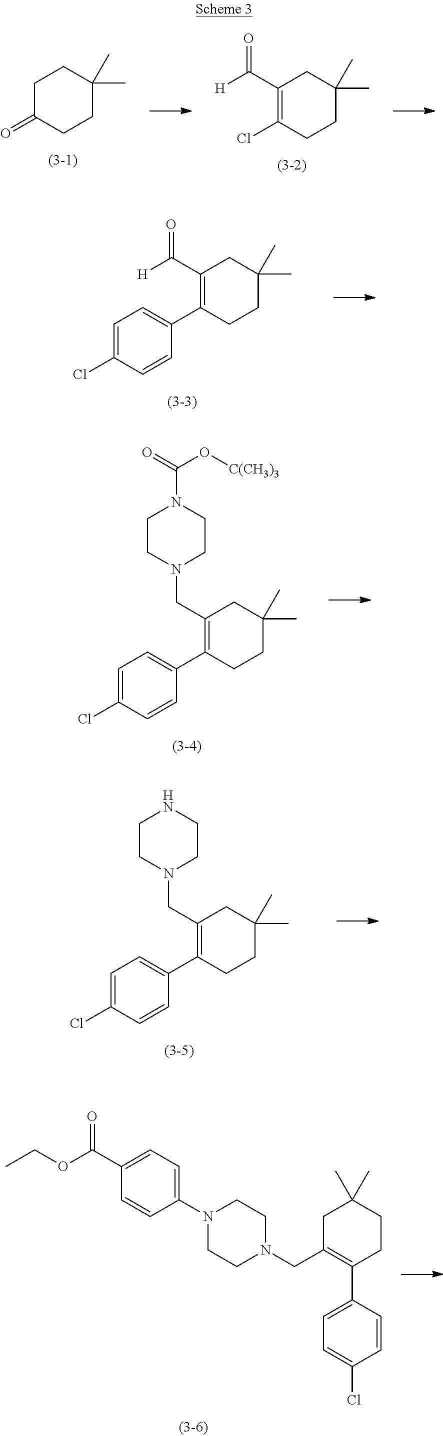

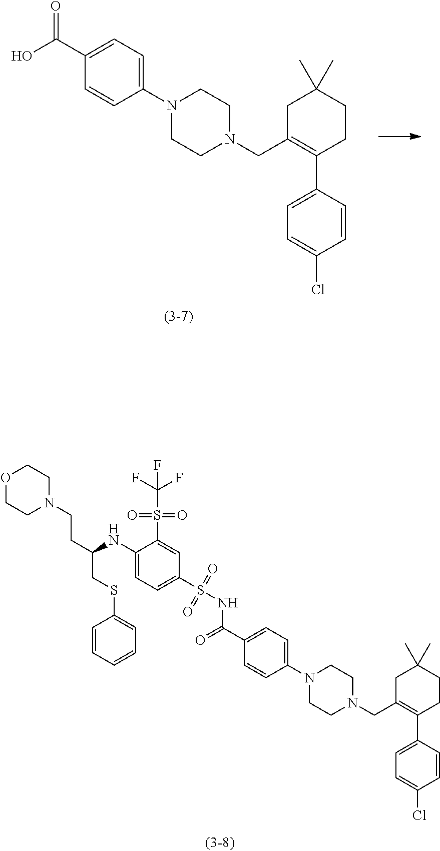 [SMILES: CC1(C)CCC(c2ccc(Cl)cc2)=C(CN2CCN(c3ccc(C(=O)O)cc3)CC2)C1 ; CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

As shown in Scheme 3, a compound of formula (3-8) can be prepared from a compound of formula (3-1). A compound of formula (3-1), 4,4-dimethylcyclohexanone, can first be treated with phosphorus oxychloride and N,N-dimethylformamide in chilled a solvent such as chilled dichloromethane. Subsequent treatment with aqueous sodium acetate and sodium chloride and then with aqueous potassium phosphate tribasic and sodium chloride provides the compound of formula (3-2). A compound of formula (3-2) can be reacted with 4-chlorophenylboronic acid or other similar coupling partner under Suzuki reaction conditions to give a compound of formula (3-3). A compound of formula (3-3) can be reacted with tert-butyl piperazine-1-carboxylate under reductive amination conditions to give a compound of formula (3-4). Subsequent protecting group removal under acidic conditions in heated isopropyl alcohol converts a compound of formula (3-4) to a compound of formula (3-5). A compound of formula (3-5) can be reacted with ethyl 4-fluorobenzoate in the presence of a base such as 1,8-diazabicyclo[5.4.0]undec-7-ene in a solvent such as heated sulfolane to supply a compound of formula (3-6). Saponification with sodium hydroxide in a heated mixture of ethanol and water transforms a compound of formula (3-6) to a compound of formula (3-7). A compound of formula (3-7) can be coupled with a compound of formula (2-7) under amide bond forming reaction conditions such as 1-ethyl-3-[3-(dimethylamino)propyl]-carbodiimide hydrochloride, N,N-dimethylpyridin-4-amine, and triethylamine in a warmed solvent such as ethyl acetate to give a compound of formula (3-8).

Unless otherwise indicated, the organic solvents used in the processes provided herein may be selected from those commercially available or otherwise known to those skilled in the art. Appropriate solvents for a given reaction are within the knowledge of the skilled person and include mixtures of solvents. Examples of organic solvents provided herein for use include but are not limited to: pentane, hexane, heptane, cyclohexane, methanol, ethanol, 1-propanol, isopropanol, 1-butanol, 2-butanol, tert-butanol, 2-butanone, dichloromethane, chloroform, carbon tetrachloride, 1,2-dichloroethane, tetrahydrofuran (THF), dimethylformamide (DMF), hexamethylphosphoramide (HMPA), N-methyl-2-pyrrolidinone (NMP), dimethyl sulfoxide (DMSO), sulfolane, nitromethane, acetone, acetic acid, acetonitrile, ethyl acetate, isopropyl acetate, diethyl ether, diethylene glycol, glyme, diglyme, petroleum ether, dioxane, methyl tert-butyl ether (MTBE), benzene, toluene, xylene, pyridine, 2-methyltetrahydrofuran, and mixtures thereof.

In some embodiments, an organic solvent used in the processes provided herein is an aprotic organic solvent. As provided herein, an aprotic solvent is a solvent that does not contain an acidic hydrogen atom or a hydrogen atom that is capable of hydrogen bonding (e.g., is not bound to an oxygen or a nitrogen atom). The aprotic organic solvent may be selected from the group consisting of dichloromethane, chloroform, acetone, acetonitrile, tetrahydrofuran (THF), dimethylformamide (DMF), hexamethylphosphoramide (HMPA), N-methyl-2-pyrrolidinone (NMP), dimethyl sulfoxide (DMSO), sulfolane, ethyl acetate, isopropyl acetate, diethyl ether, dioxane, nitromethane, pyridine, toluene, 2-methyltetrahydrofuran, and mixtures thereof. In some embodiments, the aprotic organic solvent is THF. In some embodiments, the aprotic organic solvent is DMF. In some embodiments, the aprotic organic solvent is acetonitrile.

As will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein. Common deprotection methods include treating with acids, such as, but not limited to, hydrobromic acid, hydrochloric acid, sulfuric acid, phosphoric acid, or acetic acid. The above non-limiting list of acids may be further utilized in the synthesis of compounds of this disclosure. Common deprotection methods include treating with base, such as, but not limited to, sodium hydroxide, potassium hydroxide, sodium carbonate, sodium bicarbonate, potassium carbonate, potassium bicarbonate. The above non-limiting list of bases may be further utilized in the synthesis of compounds of this disclosure.

Conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein, hereby incorporated by reference in its entirety. Common deprotection methods include treating with acids, such as, but not limited to, hydrobromic acid, hydrochloric acid, sulfuric acid, phosphoric acid, or acetic acid. The above non-limiting list of acids may be further utilized in the synthesis of compounds of this disclosure. Common deprotection methods include treating with base, such as, but not limited to, sodium hydroxide, potassium hydroxide, sodium carbonate, sodium bicarbonate, potassium carbonate, potassium bicarbonate. The above non-limiting list of bases may be further utilized in the synthesis of compounds of this disclosure.

Protecting groups for C(O)OH moieties include, but are not limited to, acetoxymethyl, allyl, benzoylmethyl, benzyl, benzyloxymethyl, tert-butyl, tert-butyldiphenylsilyl, diphenylmethyl, cyclobutyl, cyclohexyl, cyclopentyl, cyclopropyl, diphenylmethylsilyl, ethyl, para-methoxybenzyl, methoxymethyl, methoxyethoxymethyl, methyl, methylthiomethyl, naphthyl, para-nitrobenzyl, phenyl, n-propyl, 2,2,2-trichloroethyl, triethylsilyl, 2-(trimethylsilyl)ethyl, 2-(trimethylsilyl)ethoxymethyl, triphenylmethyl and the like.

Protecting groups for C(O) and C(O)H moieties include, but are not limited to, 1,3-dioxylketal, diethylketal, dimethylketal, 1,3-dithianylketal, O-methyloxime, O-phenyloxime and the like.

Protecting groups for NH moieties include, but are not limited to, acetyl, alanyl, benzoyl, benzyl(phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, formyl, methanesulfonyl, methoxyacetyl, 4-methoxybenzyloxycarbonyl, para-methoxybenzyl, methoxycarbonyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

### 2. Definitions

As provided herein, a “tertiary amine base” refers to an amine that is substituted with three alkyl groups. Examples of tertiary amine useful in the synthesis of compounds in this disclosure include, but are not limited to trimethylamine, triethylamine (TEA), N,N-diisopropylethylamine, N,N-dimethylpyridin-4-amine, 1,1,3,3-tetramethylguanidine (TMG), 2-tert-butyl-1,1,3,3-tetramethylguanidine, 1,5,7-triazabicyclo[4.4.0]dec-5-ene, 7-methyl-1,5,7-triazabicyclo[4.4.0]dec-5-ene, or 1,8-diazabicyclo[5.4.0]undec-7-ene.

As is known in the art, an “oxidant” or an “oxidizing agent” is a compound that oxidizes other agents by accepting their electrons. Examples of oxidants useful in the synthesis of compounds in this disclosure include, but are not limited to orthoperiodic acid (H₅IO₆), sodium periodate (NaIO₄), OXONE® (potassium peroxymonosulfate, KHSO₅), and sodium bromate

As is known in the art, a “reducing agent” or “antioxidant” is a compound that reduces other agents by donating electrons. Examples of reducing agents useful in the synthesis of compounds in this disclosure include, but are not limited to, sodium metabisulfite and sodium thiosulfate.

A “photoredox catalyst” or a “photoredox sensitizer” is a compound that absorbs light to give redox activated excited states. In the excited state, the photoredox catalyst is capable of carrying out redox (reduction or oxidation) reactions. Examples of photoredox catalysts useful in the synthesis of compounds in this disclosure include, but are not limited to, tris(2,2′-bipyridyl)dichlororuthenium(II) hexahydrate (Ru(bpy)₃Cl₂(H₂O)₆), (2,2′-bipyridine)bis(2-phenylpyridinato)iridium(III) hexafluorophosphate, and tris[5-fluoro-2-(2-pyridinyl-kN)phenyl-kC]iridium(III) Ir(p-F-ppy)₃.

An “alkylation agent” is a compound capable of adding an alkyl group to another compound. Alkylation agents add alkyl groups having a number of carbon atoms varying from one carbon atom (a methylating agent) up to about 12 carbon atoms, such as from one carbon atom to about 6 carbon atoms, such as one carbon atom (a methylating agent), two carbon atoms (an ethylating agent), three carbon atoms, or four carbon atoms. Examples of alkylation agents useful in the synthesis of compounds in this disclosure include trifluoromethylation agents, such as, but are not limited to, trifluoroiodomethane (CF₃I), trifluoromethyltrimethylsilane (CF₃SiMe₃), trifluoromethane (CF₃H), and trifluoromethanesulfonyl chloride (CF₃SO₂Cl). The products obtained by any of the synthetic processes provided above may be recovered by conventional means, such as evaporation or extraction, and may be purified by standard procedures, such as distillation, recrystallization, or chromatography.

When describing navitoclax, the term “free base” or “freebase” refers to navitoclax compound as distinct from any salt thereof, while recognizing that navitoclax is, strictly speaking, zwitterionic at physiological pH and thus does not always behave as a true base.

The products obtained by any of the processes provided herein may be recovered by conventional means, such as evaporation or extraction, and may be purified by standard procedures, such as distillation, recrystallization or chromatography

### Examples

In order that the invention described herein may be more fully understood, the following examples are set forth. The synthetic examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General schemes relating to methods of making exemplary compounds of the invention are additionally described in the section entitled Methods of Making Exemplary Compounds.

Unless indicated otherwise, compounds were characterized by HPLC and ¹H NMR analysis and used in later reactions with or without purification. ¹H NMR analysis was performed at 400 MHz unless otherwise indicated. Unless specified otherwise, product yield/purity was determined by weight, qNMR, and/or HPLC analysis.

Compounds of the following examples and as shown in Schemes 1 to 3 above were named using Chemdraw® Ultra, ChemDraw® Professional, or Advanced Chemistry Development Name 2020.1.2 software. In addition to the abbreviations described above with respect to the schemes provided herein, the following abbreviations are used in the Examples:

### Abbreviations

Bpy for 2,2′-bipyridiyl; CI for chemical ionization; CSTR for continuous stirred tank reactor; DMSO for dimethyl sulfoxide; DSC for differential scanning calorimetry; ESI for electrospray ionization; GC for gas chromatography; HPLC for high performance liquid chromatography; HRMS for high resolution mass spectrum; i.d. for internal diameter; KF for Karl Fischer titration; mp for melting point; MS for mass spectrum; NMR for nuclear magnetic resonance; OD or o.d. for outside diameter; ppm for parts per million; rpm for revolutions per minute; TMG for tetramethylguanidine; UV for ultraviolet; and v:v for volume:volume.

### Example 1. 1-chloro-2-[(trifluoromethyl)sulfanyl]benzene (1-2) (Continuous Tank Stirred Reactor)

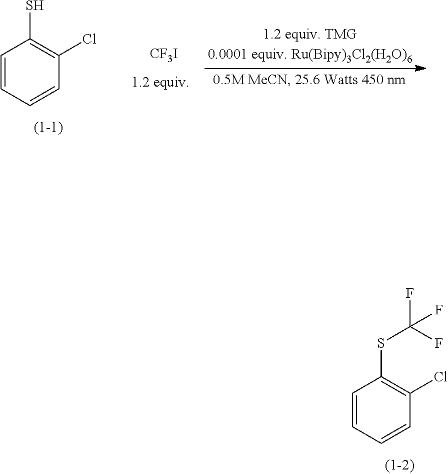

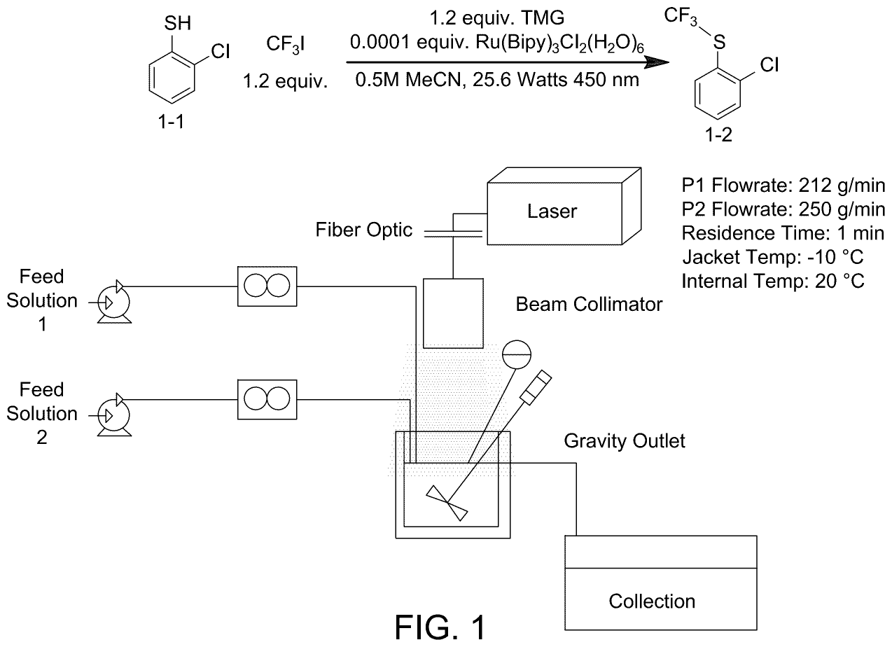

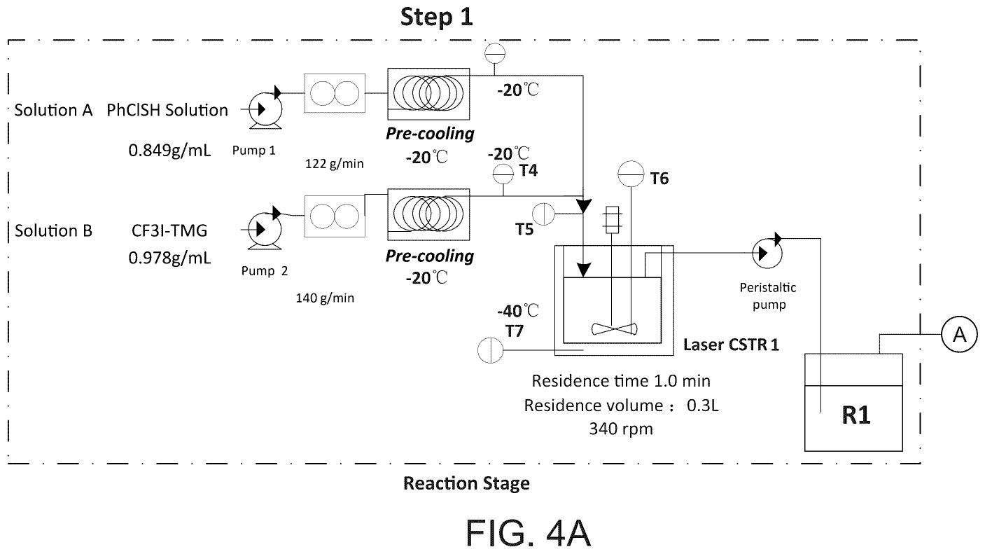

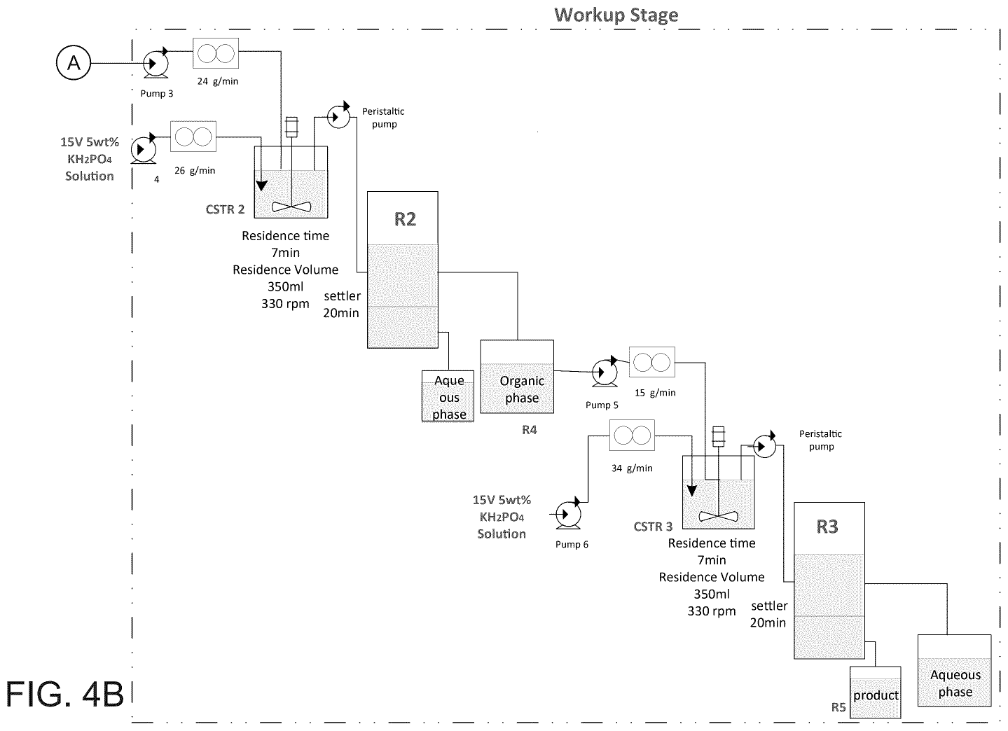

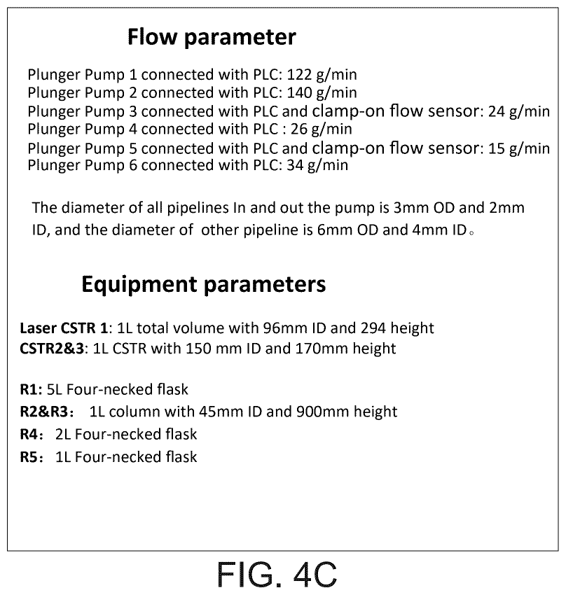

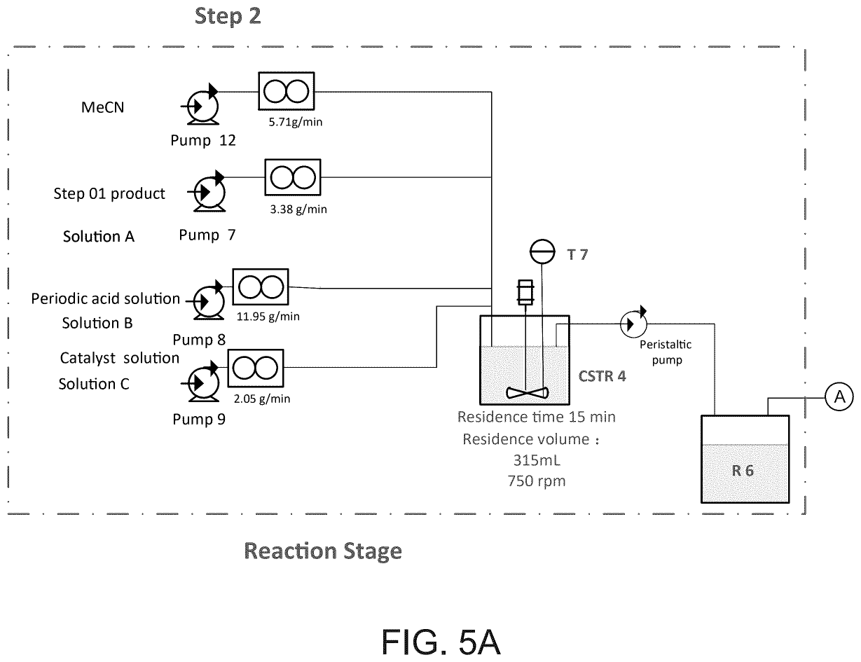

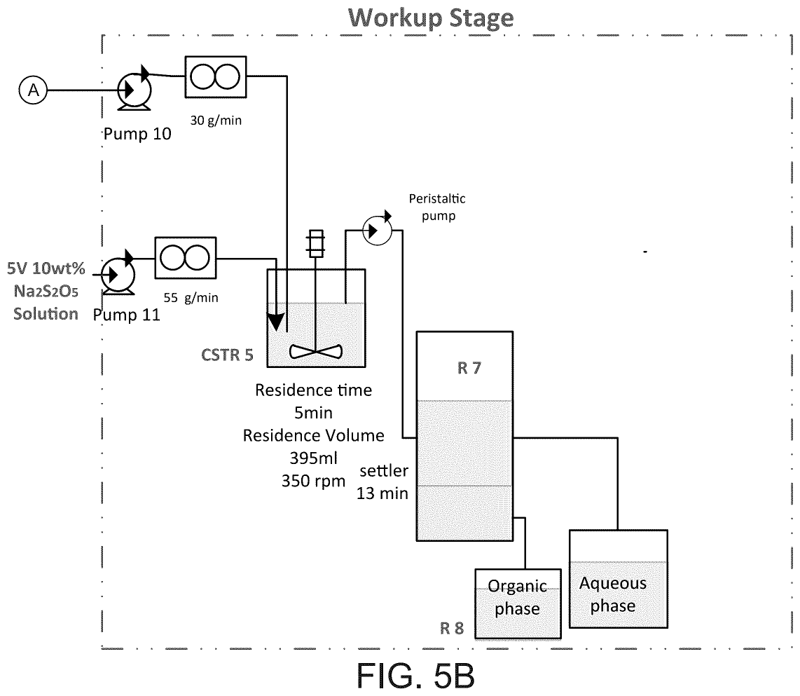

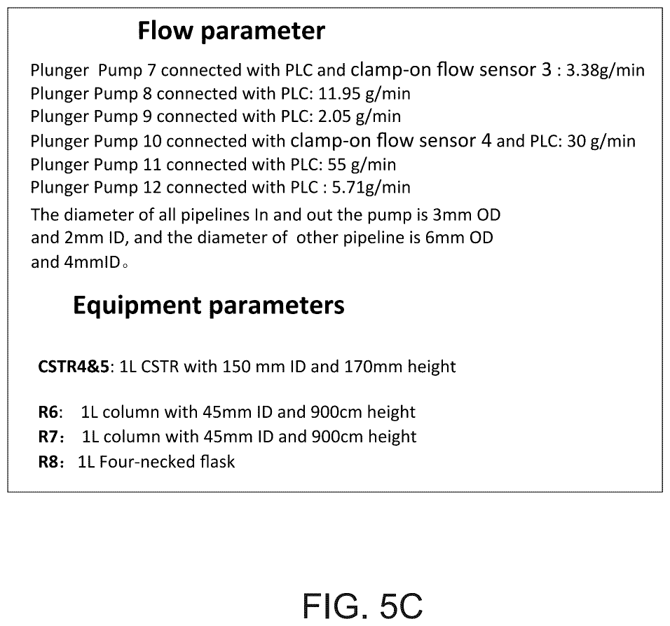

The reaction to prepare 1-chloro-2-[(trifluoromethyl)sulfanyl]benzene (1-2) from 2-chlorobenzene-1-thiol (1-1) by a photoredox flow reaction is depicted in FIGS. 1, 2, and 4A-4C. Table 1 below provides manufacturing equipment conditions for Examples 1 and 2, with reference to FIGS. 4A-4C and 5A-5C.

### Feed Solution 1 Preparation:

Feed Solution 1 (See FIG. 1) was prepared as follows. To a 10 L media bottle was added 518 mg of tris(2,2′-bipyridyl)dichlororuthenium(II) hexahydrate (Ru(bpy)₃Cl₂(H₂O)₆, 0.0001 equiv., 0.69 mmol). This was dissolved with 6.2 L of acetonitrile and the solution was stirred until complete dissolution was observed (˜4 minutes). The solution was then sparged with nitrogen (20 mL/minute) for 30 minutes. 2-Chlorobenzene-1-thiol (1-1) was then added (785 mL, 6.915 mol, 1 equivalent). The density of the solution was measured at 0.85 g/mL. A target flowrate of 212 g/minute was calculated for this solution. Feed Solution 1 was cooled to −10° C. internal temperature and held. Feed Solution 1 is depicted as Solution A in FIG. 4A.

### Feed Solution 2 Preparation:

Feed Solution 2 (See FIG. 1) was prepared as follows. To a 10 L media bottle was added 1,1,3,3-tetramethylguanidine (TMG, 1.04 L, 8.29 mol, 1.2 equivalents). This was dissolved in acetonitrile (5.2 L) and cooled to 0° C. A polytetrafluoroethylene line was added and CF₃I gas (1630 g) was added from a tared gas cylinder over ˜1 hour. The density of this solution was recorded at 0.998 g/mL. A target flowrate of 250 g/minute was calculated for this solution. Feed Solution 2 was cooled to −10° C. internal temperature and held. TMG forms a stable complex with CF₃I, and it can be readily removed during the workup via acidic washes. In preparation of Feed Solution 2, the complex was formed in situ by dissolving CF₃I gas in acetonitrile in which the TMG was previously dissolved. Feed Solution 2 is depicted as Solution B in FIG. 4A.

### Continuous Stir Tank Reactor:

As shown in FIG. 1 and FIG. 4A, Feed Solutions 1 and 2 were connected to gear pumps and mass flow meters via ¼″ OD polytetrafluoroethylene tubes. The mass flow meters were in turn connected to a 500 mL laser continuous stirred tank reactor (CSTR) diagram shown in FIG. 1 and FIG. 4A. The CSTR features a gravity outlet at 500 mL and mechanical stirring and internal thermocouple. The jacket on the CSTR was equilibrated at −10° C. prior to initiating the flows. The laser was connected via sealed beam collimater. To prevent vapor condensation on the lens, a 15 mL/minute flow of nitrogen was maintained in the headspace of the reactor for the entire run. See also FIG. 4C for flow and equipment parameters.

### Reaction:

As shown in FIG. 1 and FIG. 4A, the continuous stirred tank reactor (CSTR) was charged with a solution of 0.05 mM tris(2,2′-bipyridyl)dichlororuthenium(II) hexahydrate (Ru(bpy)₃Cl₂(H₂O)₆) in acetonitrile and equilibrated to −9° C. internal temperature when the 450 nm laser was started and set to 25.0 W output power. Once the laser was equilibrated (˜1 minute), the flow was initiated with Feed Solution 1 pumping at 212 g/minute (+/−1 g/minute) and Feed Solution 2 was initiated at 250 g/minute (+/−2 g/minute). The internal temperature rose to 18° C. quickly and gradually rose after that to 20° C. After 2 minutes the mixture resulting from the combination of Feed Solutions 1 and 2 and the solution in the CSTR were diverted to the collection vessel.

After ˜24 minutes, the Feed Solutions were down to ˜10 mL remaining. The pumps were stopped, the laser was stopped, and the reactor solution pumped out into the collection vessel.

Results: 99.2% Conversion, 96% Assay Yield (Corrected for loss on start-up).

### Workup:

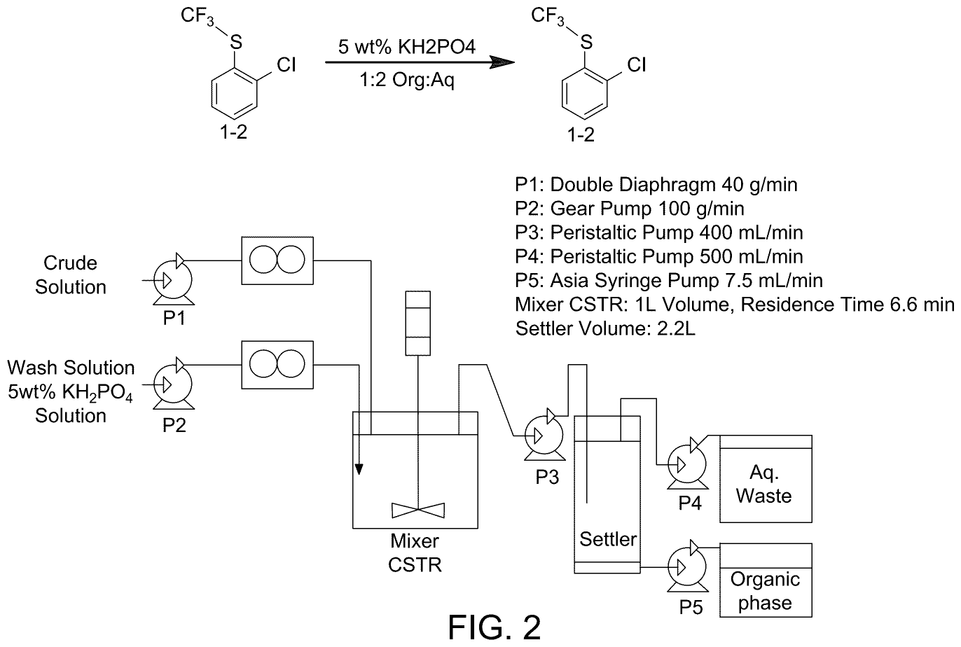

The crude solution was washed and collected in an organic phase, as shown in FIG. 2 and in Workup Stage in FIG. 4B. See also FIG. 4C for flow and equipment parameters.

### Crude Solution Preparation:

Crude Solution (See FIG. 2 and “A” in FIG. 4B) was prepared as follows. Crude 1-chloro-2-[(trifluoromethyl)sulfanyl]benzene (1-2) containing stream from the previous step and connected to the mixer CSTR via ¼″ OD polytetrafluoroethylene tubing. The density was 0.917 g/minute. The solution was connected to mixer via ⅛″ OD polytetrafluoroethylene tubing via a double diaphragm pump and mass flow meter. The target flow rate 40 g/minute.

### Wash Solution Preparation:

Wash Solution (See FIG. 2 and “KH₂PO₄ solution” FIG. 4B) was prepared as follows. 20 L of was solution comprising 5 weight % KH₂PO₄ was prepared. Pumping was provided by a gear pump. Density was 1.03 g/mL. Targe flow rate 100 g/minute.

### Mixer CSTR:

As shown in FIG. 2 and FIG. 4B, a 1 L jacketed reactor with 2 pitch blade impellors. The jacket temperature was set to 20° C. Inlets were placed on opposite sides near the bottom of the reactor. Stirring was 400 rpm. The outlet was a continuously pumped dip-tube set at a precalibrated height corresponding to 1 L total volume. The outlet was pumped actively with a peristaltic pump and fed directly into the settler as shown in the schematic.

The settler was a 2 L graduated cylinder with outlets fixed at the bottom and at the 2.2 L mark. The bottom outlet collected the organic phase. The ratio of aqueous phase (Aq. waste in FIG. 2) to organic phase was about 20:1. The organic phase contained approximately 50 weight % product.

A second wash was executed as a repeat of the original conditions.

### Example 2. 1-chloro-2-(trifluoromethanesulfonyl)benzene (1-3)

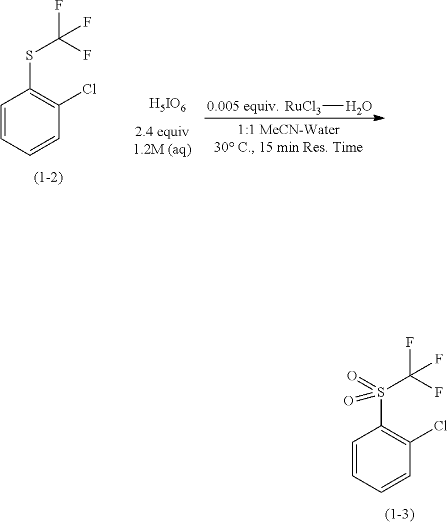

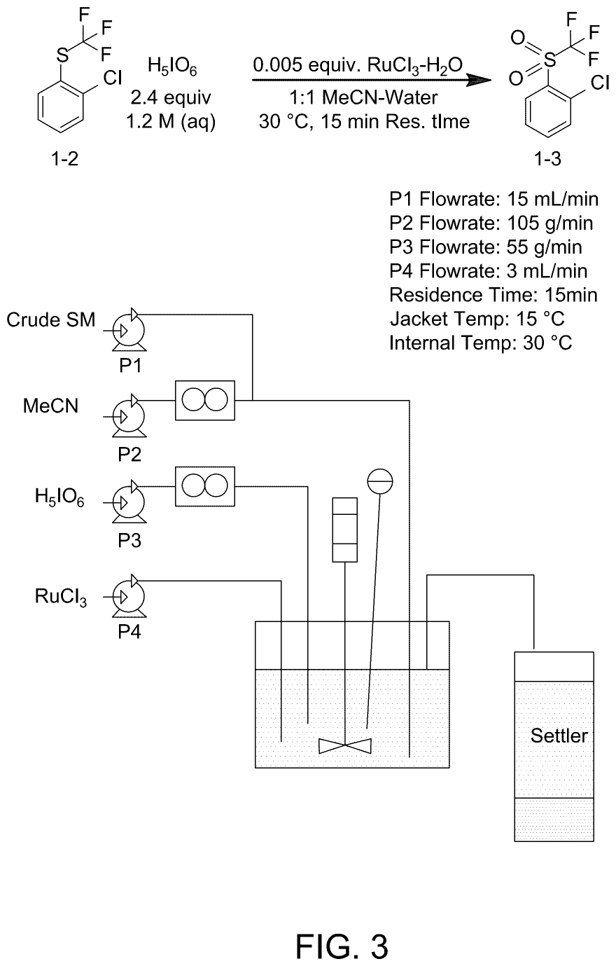

The reaction to prepare 1-chloro-2-(trifluoromethanesulfonyl)benzene (1-3) from 1-chloro-2-[(trifluoromethyl)sulfanyl]benzene (1-2) is depicted in FIGS. 3 and 5A-5C. Table 1 below provides manufacturing equipment conditions for Examples 1 and 2, with reference to FIGS. 4A-4C and 5A-5C.

### Solution 1 Preparation:

Solution 1 (Crude SM in FIG. 3 and “Solution A” in FIG. 5A) was prepared as follows. Material was carried over from the photoredox flow and continuous workup of Example 1. The total volume was approximately 1.5 L, and the density of the composition was 1.13 g/mL. Solution 1 contained approximately 61 weight % 1-chloro-2-[(trifluoromethyl)sulfanyl]benzene (1-2). The target flowrate was 15 mL/minute. This solution was connected to the reactor via a Syrris Asia pump.

Solution 2 Preparation (See FIG. 3 H₅IO₆):

Orthoperiodic acid (H₅IO₆ in FIG. 3 and Solution B in FIG. 5A) (2660 g, 2.4 equivalents, 11.67 mol) was dissolved in 7 L of deionized water in a 10 L media bottle. Dissolution took approximately 30 minutes with overhead stirring. The density of the solution was 1.22 g/mL. The target flowrate was 105 g/minute. This solution was connected to the reactor via a double diaphragm pump and mass flow meter.

### Solution 3 Preparation:

Pure acetonitrile (“MeCN” in FIG. 3 and FIG. 5A) was transferred to a 5 L media bottle. This solution was periodically refilled during the run. The density was 0.787 g/mL. The target flowrate was 55 g/minute. This solution was connected to a gear pump and mass flow meter and then directly connected to the feed line of Solution 1 via an impinging T providing in-line dilution.

### Solution 4 Preparation:

RuCl₃—H₂O (RuCl₃ in FIG. 3 and Solution C in FIG. 5A) (5.48 g) was dissolved in water (300 mL) and kept under nitrogen. The density was 1.003 g/mL. The target flowrate was 3 mL/minute. This solution was connected to the reactor via ⅛ inch polytetrafluoroethylene tubing.

### Reactor:

A 3 L jacketed reactor was used with a fixed dip tube outlet which was actively pumped to control the level at 2.7 L for a target residence time of 15 minutes. All feeds were fed via dip tubes made of SS316 to allow them to be fixed in the reactor. Each feed dip tube was placed at the bottom of the reactor as far apart as possible. Stirring was mechanical via pitched blade impellor (3 cm) and secondary impellor (3 cm) placed 4 cm up the shaft. Stir rate was 700-750 rpm during the run. See FIG. 5A. See also FIG. 5C for flow and equipment parameters.

### Reaction:

As shown in FIG. 3, the reactor was stirred at 700 rpm and the jacket temperature set to 5° C. The reactor was charged with acetonitrile (100 mL) and the pumps started. There was no diversion of the collection but all of the material was collected. Collection was into a 20 L carboy container. Target mass flow rates were equilibrated within 30 seconds of start-up. The internal temperature equilibrated at 30.5° C. The product stream was collected in a 10 L media bottle which was used as a settler and the bottom aqueous layer was pumped off.

### Product Workup:

### Feed Solution 1:

The separated product stream (“A” in FIG. 5B) from the oxidation was approximately 6 L in volume and had a density of 0.95 g/mL. This solution was connected to the mixer CSTR via gear pump and mass flow meter which led into a dip tube placed near the bottom of the reactor opposite the dip tube of Feed Solution 2. Target flowrate was 95 g/minute.

### Feed Solution 2:

Sodium metabisulfite (200 g) (“Na₂S₂O₅ solution” in FIG. 5B) was dissolved in 4 L of deionized water with stirring. The resulting solution was connected to the mixer CSTR via double diaphragm pump and mass flow meter. Target flowrate was 48 g/minute.

### Mixer CSTR:

1 L Jacketed reactor with 2 pitch blade impellors space 3 cm apart. Jacket temperature was set to 20° C. Inlets were placed on opposite sides near the bottom of the reactor. Stirring was 350 rpm to 400 rpm. The outlet was a continuously pumped dip tube set at a precalibrated height corresponding to 750 mL of total volume. The outlet was actively pumped with a peristaltic pump and fed directly into the settler as shown in FIG. 5B.

### Distillation:

The crude organic fraction from the settler was transferred into 1 L flask, and distilled at 80° C. oil bath temperature under 10 mbar vacuum to remove acetonitrile and water, then distilled at 120° C. oil bath temperature under vacuum at 3-7 mbar to collect 92-101° C. fraction to provide the title compound 1-chloro-2-(trifluoromethanesulfonyl)benzene (1-3) (436.39 g, 86% yield). 1H NMR (500 MHZ, DMSO-d₆) δ ppm 7.83 (d, J=7.7 Hz, 1H), 7.69 (dd, J=8.1, 1.4 Hz, 1H), 7.59 (td, J=7.7, 1.7 Hz, 1H), 7.50-7.44 (m, 1H).

TABLE 1
Manufacturing Equipment Table for FIGS.
4A-4C (Step 1) and 5A-5C (Step 2)

| Equipment | Parameters |
|---|---|
| Plunger Pump 1 | Connected with PLC: 122 g/minute |
| Plunger Pump 2 | Connected with PLC: 140 g/minute |
| Plunger Pump 3 | Connected with PLC and Clamp-on |
| | flow sensor 1: 24 g/minute |
| Plunger Pump 4 | Connected with PLC: 26 g/minute |
| Plunger Pump 5 | Connected with PLC and Clamp-on |
| | flow sensor 2: 15 g/minute |
| Plunger Pump 6 | Connected with PLC: 34 g/minute |
| Plunger Pump 7 | Connected with PLC and Clamp-on |
| | flow sensor 3: 3.38 g/minute |
| Plunger Pump 8 | Connected with PLC: 11.95 g/minute |
| Plunger Pump 9 | Connected with PLC: 2.05 g/minute |
| Plunger Pump 10 | Connected with PLC and Clamp-on |
| | flow sensor 4: 30 g/minute |
| Plunger Pump 11 | Connected with PLC: 55 g/minute |
| Plunger Pump 12 | Connected with PLC: 5.71 g/minute |
| Laser CSTR | 1 L total volume with 96 mm i.d. and |
| | 294 mm height 316L CSTR with jacket |
| Laser | 445 nm, 25 W, with 8.0 cm beam |
| Magnetic stirrer | IKA stirrer with temperature sensor |
| for Laser CSTR | |
| CSTR2&3 | 1 L with 150 mm i.d. and 170 mm height |
| CSTR4&5 | 1 L with 150 mm i.d. and 170 mm height |
| Clamp-on flow sensor 1 | 26.08 mL/min (reaction mixture density: |
| (Keyence FD- series) | 0.92 g/mL) |
| Clamp-on flow sensor 2 | 15.31 mL/min (organic phase density: |
| (Keyence FD- series) | 0.98 g/mL) |
| Clamp-on flow sensor 3 | 3.25 mL/minute (organic phase density: |
| (Keyence FD- series) | 1.04 g/mL) |
| Clamp-on flow sensor 4 | 27.53 mL/minute (organic phase density: |
| (Keyence FD- series) | 1.09 g/mL) |
| R1 | 5 L four necked flask |
| R2&3 | 1 L column settler with 45 mm i.d. and |
| | 900 mm height |
| R4 | 2 L four necked flask |
| R5 | 1 L four necked flask |
| R6 | 1 L column settler with 45 mm i.d. and |
| | 900 mm height |
| R7 | 1 L column settler with 45 mm i.d. and |
| | 900 mm height |
| R8 | 1 L four necked flask |
| Pipeline | The pipeline (i.d. = 2 mm, o.d. = 3 mm) |
| | was used to connect with pump and other |
| | pipeline (i.d. = 4 mm, o.d. = 6 mm) were |
| | used to connect with CSTR and column |
| | settler. |

### Example 3. Continuous Manufacturing Procedure

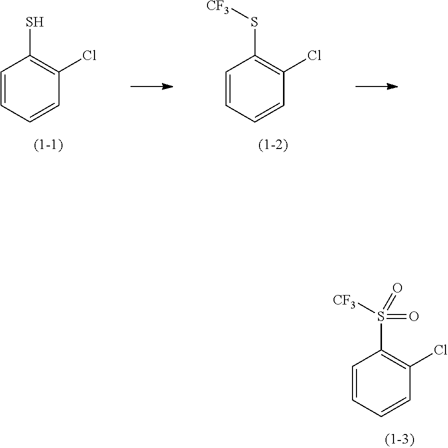 [SMILES: Sc1ccccc1Cl ; FC(F)(F)Sc1ccccc1Cl ; O=S(=O)(c1ccccc1Cl)C(F)(F)F]

This example is a procedure for the continuous manufacture of 1-chloro-2-[(trifluoromethyl)sulfanyl]benzene (1-2) from 2-chlorobenzene-1-thiol (1-1) and then 1-chloro-2-(trifluoromethanesulfonyl)benzene (1-3) from 1-chloro-2-[(trifluoromethyl)sulfanyl]benzene (1-2). This example utilizes the process flow depicted in FIGS. 4A, 4B, 5A, and 5B and the manufacturing equipment table and conditions shown in Table 1 of Example 2.

### Preparation of Solution a:

Ru(bpy)₃Cl₂·6H₂O (0.1553 g, 0.01% equiv.) and acetonitrile (1403.1 g, 1776 mL) were combined in 3 L flask A equipped with magnetic stir bar, and the solution was sparged with nitrogen gas (about 1 L/min) for 120 min. at −20° C. 2-Chlorobenzene-1-thiol (1-1) (300 g, 1.0 equiv.) was added and stirred about 10 min. under nitrogen gas protection at −5˜ 0° C.

### Preparation of Solution B (20% Excess of Solution was Prepared):

1,1,3,3-Tetramethylguanidine (344.06 g, 1.44 equiv.) and acetonitrile (1396.4 g) were combined in 3 L flask B equipped with magnetic stir bar at −20° C., and the solution was sparged with nitrogen gas (about 1 L/min) for 120 min at −20° C. CF₃I gas (585.21 g, 1.44 equiv.) was dissolved into this solution at −20° C.

Preparation of 5 wt % KH₂PO₄ Solution (5% Excess of Solution was Prepared):

KH₂PO₄ (240.98 g) and H₂O (4349 g) were combined in a 5 L flask bottle equipped with magnetic stir bar, and the solution was stirred for 15 min.

Preparation of H₅IO₆ Solution (2.3 Equiv. Based on 100% Yield for Step 1):

H₅IO₆ (1087.5 g) and H₂O (1500 g) were combined in a 2 L flask bottle equipped with magnetic stir bar at room temperature and stirred for 15 min.

Preparation of RuCl₃ Solution (0.5% Equiv. Based on 100% Yield for Step 1):

RuCl₃·4H₂O (2.90 g) and H₂O (441 g) were combined in a 500 mL flask bottle equipped with magnetic stir bar at room temperature and stirred for 15 min.

Preparation of 10 wt % Na₂S₂O₅ solution (5% excess of solution was prepared):

Na₂S₂O₅ (245.45 g) and H₂O (2209 g) were combined in a 2 L flask bottle equipped with magnetic stir bar at room temperature and stirred for 15 min.

### Setup Detail:

Pumps: Plunger pump 3&5&7&10 were connected to clamp-on flow sensor to control flow rate. Other plunger pumps together with balances were connected with PLC to control pump feeding rate.

### Flow Reaction Execution:

### Startup Protocol for Step 1 Reaction Stage:

The CSTR jacket was cooled −40° C. in advance, and the IKA magnetic stirrer was set 340 rpm and the temperature sensor was monitored the CSTR internal temperature, the nitrogen gas flow rate at the head of the solvent was 400 mL/min. The laser was turned on and set to provided 25 W of output light. Pump 2 (solution B) was started for 10 s in advance, then the pump 1 was started to feed the 2-chlorobenzene-1-thiol (1-1) solution (solution A). The two feeding solution was pre-cooled about 20 seconds in tube and then mixed via arrow mixer immersed in −20° C. coolant. And samples were collected every 3 minutes for step 1 at outlet. The crude mixture in CSTR was transferred into 5 L four neck flask by peristaltic pump to keep irradiation volume at 300 mL with 1 min residence time at −8.5˜ 3.2° C.

### Shutdown Protocol Step 1 Reaction Stage:

Upon consumption of 2-chlorobenzene-1-thiol (1-1) solution (solution A), pump 1 was turned off. Pump 2 (solution B, CF₃I-TMG solution) was fed an extra 30 seconds, and then Pump 2 was turned off. The laser was turned off 1 minute after Pump 2 was turned off.

### Workup Stage for Step 1:

The crude mixture of 1-chloro-2-[(trifluoromethyl)sulfanyl]benzene (1-2) was transferred into CSTR 2 via pump 3 controlled by PLC and clamp-on flow sensor with 24 g/min flow rate. The mixture was washed with 15 Vol. 5 wt % KH₂PO₄ solution twice, and residence time in CSTR2&3 were 7 min with 330 rpm and 20 min in settler (R2 & R3). The pump 4 and pump 5 were used for pumping 5 wt % KH₂PO₄ solution. The organic solution was at up layer of the settler after washing the first 15 Vol. 5 wt % KH₂PO₄ solution and transferring into the R4 (2 L four neck flask) via peristaltic pump. The organic phase was transferred into CSTR 3 via pump 5 controlled by PLC and clamp-on flow sensor with 15 g/min flow rate. The second washed organic layer was at the bottom of the 1 L settler (R3). The organic sampling for testing TMG residue.

### Shutdown Protocol for Workup Stage of Step 1:

When the reaction mixture from R1 (5 L four necked flask), the pump 3 was switched into acetonitrile for 1 min, and the pump 4 was stopped after pump 3 turned off. The quenched solution in CSTR 2 was stirred 7 min, then transferred into R2 (settler). The separated organic phase was then transferred into R4 (2 L four necked flask), upon consumption of the first washed organic phase in R4 (2 L four necked flask), the pump 5 was switched into acetonitrile for 1 min, and pump 6 pumped 15 V 5 wt % KH₂PO₄ solution for 1 min and stopped. The second washed mixture in CSTR 3 was stirred 7 min then transferred into R3 (settler). the separated organic phase was then transferred into R5 (1 L four necked flask).

### Startup Protocol for Step 2 Reaction Stage:

The Jacket of CSTR 4 &5 were cooled via 20-25° C. water, pump 8 (feeding H₅IO₆ solution) the and pump 9 (feeding RuCl₃ solution) were started in advance 1 min with 11.95 g/min and 2.05 g/min, respectively. Then pump 12 (feeding acetonitrile solution) with 5.71 g/min and pump 7 (feeding step 1 product solution from R5) with 3.38 g/min was started and the four feeding solution were mixed at CSTR 4 with 15-min residence time at 750 rpm and the reaction mixture temperature was ranged 25-30° C. The reaction mixture was transferred into R6 (1 L column settler) with 10-minute residence time via peristaltic pump. The separated upper layer organic phase in R6 (1 L column settler) was pumped into the CSTR 5 via pump 10. The pump 11 (feeding 5 V 10 wt % Na₂S₂O₅ solution) was started before 1 min starting pump 10. The mixture was quenched in CSTR 5 with 5-minute residence time at 25-30° C., and then transferred into R7 (1 L column setter) with 13-minute residence time via peristaltic pump. The organic layer at the bottom of R7 was transferred into R8 (1 L four necked flask).

### Shutdown Protocol for Step 2 Quenched Stage:

After step 1 product solution (pump 7) was exhausted, pump 12&8&9 were further fed for another 1 minute and then stopped. The reaction mixture in CSTR4 was further stirred for 15 minutes, then transferred into R6 via peristaltic pump, and the separated organic phase in R6 was pumped into CSTR5. When the organic phase in R6 was exhausted, pump 11 was further fed for 1 minute before stopping pump. The quenched mixture was stirred for 5 minutes and then transferred into R7 to separate organic phase.

### Distillation:

The crude product solution from R8 was transferred into 1 L flask, and distilled at 80° C. oil bath temperature under 10 mbar vacuum to remove acetonitrile and water, then distilled at 120° C. oil bath temperature under vacuum at 3-7 mbar to collect 92-101° C. fraction to provide 436.39 g product 1-chloro-2-(trifluoromethanesulfonyl)benzene (1-3) in 86% yield. 1H NMR (500 MHZ, DMSO-d₆) δ ppm 7.83 (d, J=7.7 Hz, 1H), 7.69 (dd, J=8.1, 1.4 Hz, 1H), 7.59 (td, J=7.7, 1.7 Hz, 1H), 7.50-7.44 (m, 1H).

### Continuous Manufacturing Analytical Methods

TABLE 2
Analytical method for step 1

| Column: | Waters T3 Cortecs, 100 × 4.6 mm 2.7 μm | |
|---|---|---|
| | P/N: 186008494; | |
| Column | 35° C. | |
| Temperature: | | |
| Mobile phase: | A: 0.1% HClO₄ in water | |
| | B: Acetonitrile | |
| Flow rate: | 1.5 ml/min (Ghost trap can be used if possible) | |
| Injection volume: | 5 μL | |
| Gradient: | Time(min) | % B |
| | 0.0 | 10 |
| | 10 | 90 |
| | 13 | 95 |
| | 13.1 | 10 |
| | 16 | 10 |

| | |
|---|---|
| Run time: | 16 minutes |
| Autosampler | 5° C. |
| temperature: | |
| Detection: | UV at 220 nm |
| Needle wash | Acetonitrile |
| solvent: | |

TABLE 3
Analytical method for step 2 IPC

| Column: | Ascentis express C8, 150 × 4.6 mm, | |
|---|---|---|
| | 2.7 μm P/N: 53838-U; | |
| Column | 40° C. | |
| Temperature: | | |
| Mobile phase: | A: 0.1% H₃PO₄ in water(v:v) | |
| | B: Acetonitrile | |
| Flow rate: | 1.0 ml/min (Ghost trap can be used if possible) | |
| Injection volume: | 5 μL | |
| Gradient: | Time(min) | % B |
| | 0.0 | 10 |
| | 5 | 45 |
| | 20 | 60 |
| | 23 | 95 |
| | 30 | 95 |
| | 30.1 | 10 |
| | 37 | 10 |

| | |
|---|---|
| Run time: | 37 minutes |
| Autosampler | 5° C. |
| temperature: | |
| Detection: | UV at 210 nm |
| Needle wash | Acetonitrile |
| solvent: | |

TABLE 4
Analytical method for step 2 Product

| Column: | Ascentis express C8, 150 × 4.6 mm, | |
|---|---|---|
| | 2.7 μm P/N: 53838-U; | |
| Column | 40° C. | |
| Temperature: | | |
| Mobile phase: | A: 0.1% H₃PO₄ in water(v:v) | |
| | B: Acetonitrile | |
| Flow rate: | 1.0 ml/min (Ghost trap can be used if possible) | |
| Injection volume: | 5 μL | |
| Gradient: | Time(min) | % B |
| | 0.0 | 10 |
| | 5 | 45 |
| | 20 | 60 |
| | 23 | 95 |
| | 30 | 95 |
| | 30.1 | 10 |
| | 37 | 10 |

| | |
|---|---|
| Run time: | 37 minutes |
| Autosampler | 5° C. |
| temperature: | |
| Detection: | UV at 210 nm |
| Needle wash | Acetonitrile |
| solvent: | |

### Example 4. 1-chloro-2-[(trifluoromethyl)sulfanyl]benzene (1-2) (Plug Flow Photo Reactor)

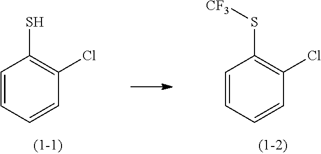 [SMILES: Sc1ccccc1Cl ; FC(F)(F)Sc1ccccc1Cl]

### Feed Solution 1 Preparation:

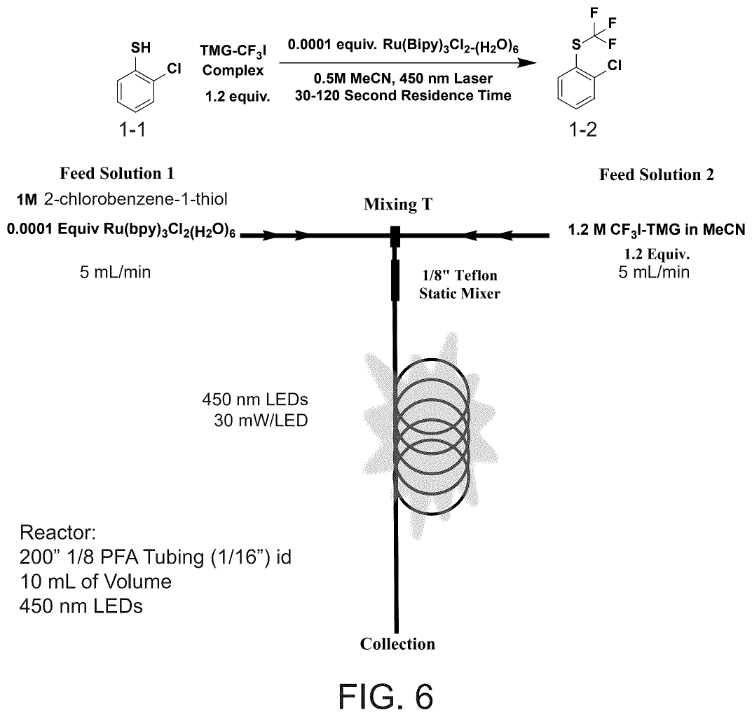

To a 2 L media bottle was charged tris(2,2′-bipyridyl)dichlororuthenium(II) hexahydrate (Ru(bpy)₃Cl₂(H₂O)₆, 135 mg, 0.0001 equivalent, 0.18 mmol). See Feed Solution 1 in FIG. 6. This was dissolved in acetonitrile (1530 mL). The solution was stirred and sparged with nitrogen for 30 minutes at ambient temperature. 2-Chlorobenzene-1-thiol (260 g, 1 equivalent, 1800 mmol) was then charged giving a 1.0 M solution of 2-chlorobenzene-1-thiol in acetonitrile. The solution was sparged again and then kept under a positive pressure of nitrogen. It was attached to a Syrris Asia pump.

### Feed Solution 2 Preparation:

To a 2 L media bottle was charged 1,1,3,3-tetramethylguanidine (250 g, 1.2 equivalents, 2159 mmol). This was dissolved in acetonitrile (1330 mL). See Feed Solution 2 in FIG. 6. This was sparged for 10 minutes and then cooled under nitrogen to 5° C. in an ice/salt bath. Once cooled and while stirring, CF₃I (423 g, 1.2 equivalents, 2159 mmol) was charged directly to the solution via a submerged polytetrafluoroethylene tube. The rate of addition was adjusted such that the internal temperature was maintained below 5° C. Prior to addition the media bottle had been tared and after dosing the gas it was weighed indicating that 430 grams of CF₃I was dissolved. This provided a 1.2 M solution of 1,1,3,3-tetramethylguanidine/CF₃I. This solution was held at 0° C. for the duration of the run and also attached to a Syrris Asia pump.

### Reactor Configuration:

See FIG. 6. The outlet of the pumps was connected to a mixing T followed by a polytetrafluoroethylene helical static mixing element to ensure good initial mixing. The residence loop was composed of 200″ of ⅛″ OD PFA (perfluoroalkoxy) tubing. This gave an estimated residence volume of 10 mL and a target residence time of 2 minutes. The T and the static mixing element were wrapped in foil to maintain the residence loop as the only irradiated reaction volume. This was wrapped around a glass pump trap and 30 mW 450 nm LEDs (light emitting diodes) were wrapped around the inside of the trap which was 1 cm distance between the reactor and the LEDs. The LEDs were wired to an adjustable controller allowing fine tuning of the output power. The power was adjusted to an estimated 15.5 mW/cm² irradiation at 1 cm distance from the LEDs, which was measured by a ThorLabs portable power meter. This reactor was run at ambient temperature or for the temperature controlled experiments, submersed in a recirculating bath.

### Flow Reaction:

See FIG. 6. With the LEDs on the correct power setting, the reaction was begun by starting each pump at 5 mL/minute and this flowrate was held for the entire flow run. After 2 minutes, a 5 μL sample was taken directly from the outlet and diluted in acetonitrile and analyzed to show complete conversion. After 7 minutes a passing sample was obtained and the product stream was then diverted to collection from waste and reaction monitored by periodic sampling of the outlet. Steady state was held for 360 minutes at which time the solution of starting material was exhausted and the pumps stopped and the reactor flushed with clean acetonitrile. The collected material was analyzed for potency and shown to contain 367.84 g of the desire product for 96% potency adjust yield.

The solution was then combined and worked up by washing with 15 volumes of a 5 weight % solution of monobasic potassium phosphate twice. The resulting solution was analyzed by HPLC and shown to contain 344.85 g of the titled compound for 90% potency adjusted yield after washing.

### Example 5. 1-chloro-2-[(trifluoromethyl)sulfanyl]benzene (1-2)

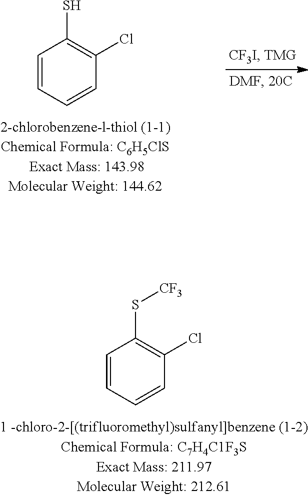 [SMILES: Sc1ccccc1Cl ; FC(F)(F)Sc1ccccc1Cl]

A solution of tetramethylguanidine (1.2 equivalents) in N,N-dimethylformamide (30 mL, 15 volumes) was cool to approximately 15° C. 2-Chlorobenzene-1-thiol (1-1) (1.6 mL, 1 equivalent) was added dropwise, maintaining the temperature <30° C. Next a CF₃I solution in N,N-dimethylformamide (3 equivalents, ˜9 mL, prepared by bubbling CF₃I into tared amount of N,N-dimethylformamide at 0° C.) was added, maintaining the reaction mixture at no more than 30° C. The reaction mixture was mixed at 15° C., and after 12 hours the reaction conversion was monitored by HPLC on a Waters™ T3 CORTECS C18, 4.6×100 mm, 2.7 μm, column eluted with 5-95% acetonitrile in 0.1% aqueous HClO₄ over 4.5 minutes and held at 95% acetonitrile in 0.1% aqueous HClO₄ for 8.0 minutes. The reaction mixture was diluted with tert-butyl methyl ether (30 mL, 15 volumes) and extracted with purified water (10 mL, 5 volumes) twice. The organic layer fraction was concentrated, and the residue was chased with tert-butyl methyl ether (10 mL, 5 volumes) at no more than 40° C., and no less than 200 mbar to give the title compound 1-chloro-2-[(trifluoromethyl)sulfanyl]benzene (1-2) (2.80 g oil, yield=85.5% (after adjusted for solvent). By 1H NMR, contained 6.6% tert-butyl methyl ether and 3.6% N,N-dimethylformamide.

### Example 6. 1-chloro-2-(trifluoromethanesulfonyl)benzene (1-3)

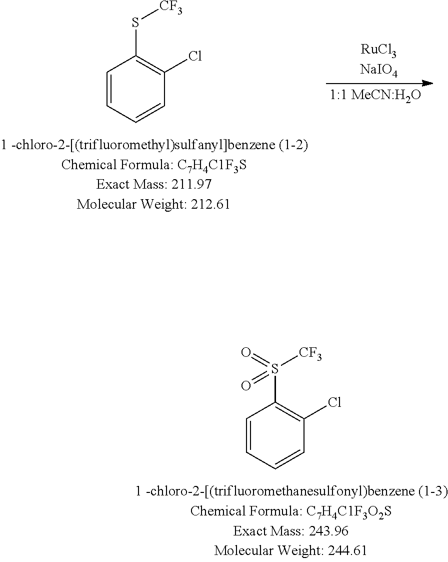 [SMILES: FC(F)(F)Sc1ccccc1Cl ; O=S(=O)(c1ccccc1Cl)C(F)(F)F]

1-Chloro-2-[(trifluoromethyl)sulfanyl]benzene (1-2) (2 g, 1 eq) was mixed in acetonitrile (15 mL, 7.5 volumes) and purified water (15 mL, 7.5 volumes), and the mixture was cooled to approximately 18° C. Ruthenium chloride (4.2 mg, 0.2 mol %) was added to the solution, followed by the addition of sodium periodate (6.0 g, 3 equivalents) in portions, maintained the temperature under 30° C. The reaction mixture was mixed for approximately 2 hours, and then monitored for reaction conversion by HPLC on a Waters™ T3 CORTECS C18, 4.6×100 mm, 2.7 μm, column eluted with 5-95% acetonitrile in 0.1% aqueous HClO₄ over 4.5 minutes and held at 95% acetonitrile in 0.1% aqueous HClO₄ for 8.0 minutes. tert-Butyl methyl ether (20 mL, 10 volumes) was added to the reaction mixture and then the mixture was quenched with 10% sodium thiosulfate (20 mL, 10 volumes). The mixture was filtered to remove salts that were rinsed with additional tert-butyl methyl ether (4 mL, 2 volumes). The layers were separated, and the organic layer was washed with purified water (10 mL, 5 volumes). The organic fraction was concentrated, and the residue was chased with tert-butyl methyl ether (10 mL, 5 volumes) at no more than 40° C., and no less than 200 mbar to give 1.60 g of the title compound 1-chloro-2-(trifluoromethanesulfonyl)benzene (1-3) (1.60 g, yield=73% (after adjusted for solvent)). By 1H NMR, oil contained 4.6% tert-butyl methyl ether and 1.1% acetonitrile.

### Example 7. 4-chloro-3-(trifluoromethanesulfonyl)benzene-1-sulfonyl chloride (1-4)

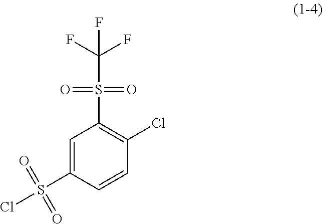 [SMILES: O=S(=O)(Cl)c1ccc(Cl)c(S(=O)(=O)C(F)(F)F)c1]

A solution of 1-chloro-2-(trifluoromethanesulfonyl)benzene (1-3) (12 g, 49.1 mmol, CAS #382-70-7) and chlorosulfonic acid (22.8 g, 196 mmol, 4.0 equivalents) was heated at 110° C. for 12 hours. The reaction mixture was cooled to 40° C. and thionyl chloride (18 g, 151 mmol, 3.1 equivalents) was added. The resultant solution was mixed at 40° C. for 4 hours. The reaction solution was cooled to 21° C. and then slowly added over 2.5 hours to 3° C. H₂O (120 mL). The resulting solids were collected by filtration and washed with 3° C. H₂O (24 mL) to afford the title compound 4-chloro-3-(trifluoromethanesulfonyl)benzene-1-sulfonyl chloride (1-4) (15.54 g, 92.5% yield). The wet cake was used in the next step without further processing. 1H NMR (400 MHZ, CDCl₃) δ ppm 8.53 (d, J=2.4 Hz, 1H), 8.35 (dd, J=8.5, 2.4 Hz, 1H), 7.95 (d, J=8.5 Hz, 1H); ¹³C NMR (101 MHz, CDCl₃) δ ppm 143.88, 143.54, 134.97, 134.73, 133.19, 132.64 (q, JCF=2 Hz) 119.78 (q, JCF=320 Hz); HRMS (ESI⁻) (Hydrolysis occurred in the ion source.) m/z calculated for C₇H₃ClF₃O₅S₂ [M−H]⁻: 322.9063; found: 322.9074.

### Example 8. 4-chloro-3-(trifluoromethanesulfonyl)benzene-1-sulfonamide (1-5)

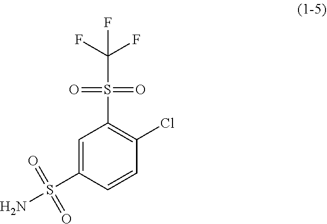 [SMILES: NS(=O)(=O)c1ccc(Cl)c(S(=O)(=O)C(F)(F)F)c1]

A solution of 4-chloro-3-(trifluoromethanesulfonyl)benzene-1-sulfonyl chloride (1-4) (15.54 g, 45.3 mmol) in isopropyl acetate (160 mL) was cooled to 0° C. Aqueous ammonia (25 weight %, 9.48 g, 139 mmol, 3.07 equivalents) was slowly added maintaining the internal temperature below 5° C. After 0.5-1 hour, H₂O (48 mL) was added to the reaction slurry. The pH was adjusted to 7-8 with 0.6 N HCl and then the mixture was warmed to 25° C. The lower aqueous layer was separated and extracted with isopropyl acetate (80 mL). The combined organic layers were washed with H₂O (48 mL). The organic fraction was concentrated under vacuum to an approximate volume of 180 mL. The concentration was continued while charging toluene to maintain an approximate volume of 180 mL until the level of isopropyl acetate was 15-20% (determined by GC or NMR). The slurry was warmed to 45° C., held for 15 minutes, then cooled to 5° C. over 1 hour. The slurry was filtered, and the collected solid was washed with 5° C. 15 weight % isopropyl acetate in toluene (24 mL). The solid was dried under vacuum at 50° C. to afford the title compound, 4-chloro-3-(trifluoromethanesulfonyl)benzene-1-sulfonamide (1-5) (12.68 g, 86.5% yield). 1H NMR (400 MHZ, DMSO-d₆) δ ppm 8.55 (d, J=1.8 Hz, 1H), 8.32 (dd, J=8.4, 2.1 Hz, 1H), 8.16 (d, J=8.4 Hz, 1H), 7.84 (s, 2H); ¹³C NMR (101 MHZ, DMSO-d₆) δ ppm 144.49, 137.81, 135.57, 134.89, 131.74, 128.85 (q, JCF=2 Hz), 119.36 (q, JCF=325 Hz); HRMS (ESI-) m/z calculated for C₇H₅ClF₃NO₄S₂ [M−H]⁻: 321.92278; found: 321.92236.

### Example 9. benzyl [(2R)-1-hydroxy-4-(morpholin-4-yl)-4-oxobutan-2-yl]carbamate (2-2)

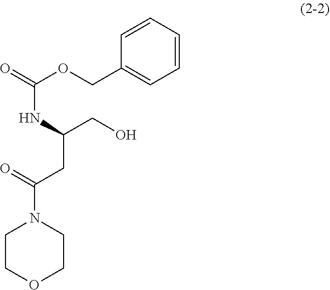 [SMILES: O=C(N[C@@H](CO)CC(=O)N1CCOCC1)OCc1ccccc1]

To a 3 L three-neck round bottom flask equipped with a mechanical stirrer, a reflux condenser and a temperature probe were charged benzyl [(3R)-5-oxooxolan-3-yl]carbamate (2-1) (123.16 g, 0.524 mol, 1.00 equivalent, CAS #118399-28-3) and 2-methyltetrahydrofuran (200 g). To the above stirred slurry was added morpholine (92.00 g, 1.056 mol, 2.0 equivalents) at ambient temperature, followed by addition of 2-methyltetrahydrofuran (10 g) as a rinse. The reaction mixture was heated to an internal temperature of approximately 65° C. and stirred for 20 hours. The reaction mixture was then cooled down 0-5° C. and quenched by slow addition of 26% brine solution (492 g) while maintaining an internal temperature≤20° C. The pH value of the quenched reaction mixture was adjusted stepwise to 7.0-8.0 with 15% hydrochloric acid and then to 5.0-6.0 with 1.8% hydrochloric acid while maintaining an internal temperature≤10° C. The mixture was allowed to warm up to 20-25° C. and stirred for 15 minutes. The upper organic phase was separated. The lower aqueous phase was back extracted with 2-methyltetrahydrofuran (493 g). The combined organic fractions were washed with 26% brine solution (246 g) and concentrated to a weight of approximately 369 g under vacuum. The concentrated solution was then chased under vacuum with acetonitrile (985 g×3) while maintaining a weight of approximately 369 g. The solution was diluted with acetonitrile (985 g) and a sample was analyzed for water content (KF≤0.1%). The solution was filtered through a layer of diatomaceous earth to remove inorganic salts. The pad and collected solids were rinsed with acetonitrile (62 g). The filtrate (1400 g) was further diluted with acetonitrile to a final weight (1877 g) that is equal to 9.0 weight % of the title compound as a solution in acetonitrile, assuming 100% yield for the reaction. This solution was used directly in next step without further process. However, an analytical sample was obtained by silica gel column purification and removal of solvents to give the title compound, benzyl [(2R)-1-hydroxy-4-(morpholin-4-yl)-4-oxobutan-2-yl]carbamate (2-2). MS (ESI⁺) m/z 323.2 [M+H]⁺; ¹H NMR (400 MHZ, DMSO-d₆) δ ppm 7.35 (m, 5H), 7.02 (d, 1H, J=8.4 Hz), 6.27 (s, 1H), 5.00 (s, 2H), 3.86 (m, 1H), 3.2-3.6 (m, 10H), 2.52 (dd, 1H, J=15.2, 5.7 Hz), 2.42 (dd, 1H, J=15.2, 7.7 Hz).

### Example 10. benzyl [(2R)-4-(morpholin-4-yl)-4-oxo-1-(phenylsulfanyl)butan-2-yl]carbamate (2-4)

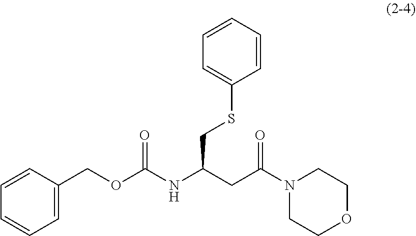 [SMILES: O=C(N[C@@H](CSc1ccccc1)CC(=O)N1CCOCC1)OCc1ccccc1]

To a 5 L jacket flask equipped with a mechanical stirrer, temperature probe and additional funnel was charged the above benzyl [(2R)-1-hydroxy-4-(morpholin-4-yl)-4-oxobutan-2-yl]carbamate (2-2) solution (1877 g, 9.0 weight %, ˜0.524 mol, 1.00 equivalent). The solution was cooled down −15° C. Triethylamine (72.2 g, 0.714 mol, 1.35 equivalents) was added slowly via an additional funnel and keeping the internal temperature≤−10° C. The funnel was rinsed with acetonitrile (10 g), and the rinse was transferred to the reactor. A pre-cooled solution of methanesulfonyl chloride (75.0 g, 0.655 mol., 1.25 equivalents) in acetonitrile (225 g) was then added slowly via the additional funnel while maintaining an internal temperature of <−5° C. The funnel was rinsed with acetonitrile (50 g), and the rinse was transferred to the reactor. The resulting mixture was stirred at −10±5° C. for 30 minutes. Sodium benzenethiolate (117.0 g, 0.886 mol., 1.69 equivalents) was added in three portions over 60 minutes at an internal temperature of −10±5° C. The resulting reaction mixture was adjusted to 20-25° C. over 2 hours and stirred for no less than 20 hours. The reaction mixture was concentrated under vacuum to a weight of ˜ 980 g. Water (1680 g) was added, and the solution was concentrated under vacuum to a weight of ˜ 1480 g. Toluene (1720 g) was added, and the internal temperature of the mixture was adjusted to 20-25° C. The pH of the mixture was then adjusted to 9.0-9.5 with aqueous 10% KOH solution while maintaining an internal temperature of 20-25° C. The mixture was stirred for 15 minutes and filtered through a layer of diatomaceous earth to remove any residual solids. The solids were rinsed with toluene (170 g). The upper organic phase was separated and washed with water (1690 g×2) and 20% brine solution (1690 g), sequentially. The upper organic phase was concentrated under vacuum and chased with toluene (825 g×2) to a weight of ˜ 490 g. The concentrated solution was diluted with toluene (825 g) and filtered through a layer of diatomaceous earth. The filtrate was concentrated under vacuum to a final weight of ˜ 566 g solution that is equal to 38.3 weight % of the product, assuming 100% yield for the reaction. The solution was used directly in the next step. An analytical sample was obtained by silica gel column purification to give the title compound, benzyl [(2R)-4-(morpholin-4-yl)-4-oxo-1-(phenylsulfanyl)butan-2-yl]carbamate (2-4). MS (ESI⁺) m/z 415.2 [M+H]⁺; ¹H NMR (400 MHZ, CDCl₃) δ ppm 7.25-7.45 (m, 9H), 7.18 (t, 1H, J=7.3 Hz), 6.04 (d, 1H, J=8.7 Hz), 5.08 (s, 2H), 4.12 (m, 1H), 3.45-3.65 (m, 6H), 3.35 (dd, 1H, J=14.2, 5.5 Hz), 3.30 (m, 2H), 3.19 (dd, 1H, J=14.2, 8.5 Hz), 2.86 (dd, 1H, J=16.2, 4.6 Hz), 2.52 (dd, 1H, J=16.2, 5.5 Hz).

### Example 11. (3R)-3-amino-1-(morpholin-4-yl)-4-(phenylsulfanyl)butan-1-one (2-5)

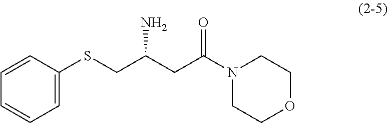 [SMILES: N[C@@H](CSc1ccccc1)CC(=O)N1CCOCC1]

To a 5 L jacket flask equipped with a mechanical stirrer, temperature probe and additional funnel was charged the above toluene solution of benzyl [(2R)-4-(morpholin-4-yl)-4-oxo-1-(phenylsulfanyl)butan-2-yl]carbamate (2-4) (566 g, 38.3 weight %, ˜0.524 mol, 1.00 equivalent), followed by addition of acetic acid (57 g) as a co-solvent. The mixture was cooled down to an internal temperature of 0-5° C. 30% Hydrobromic acid in acetic acid (615.0 g, 184.5 g HBr, 2.28 mol, 4.35 equivalents) was added slowly via the additional funnel while maintaining an internal temperature of no more than 20° C. The reaction mixture was adjusted to 20-25° C. and stirred for 1 hour. The reaction mixture was then cooled down to 0-5° C. and quenched by slow addition of water (1320 g) at an internal temperature of ≤20° C. The quenched reaction mixture was allowed to warm up to 20-25° C. and stirred for 15 minutes. The lower aqueous solution (product phase) was separated and washed with toluene (1145 g×2). The aqueous solution was then cooled down to 0-5° C., followed by addition of dichloromethane (1045 g). The pH of the mixture was adjusted initially to 7.0-8.0 with aqueous 50% KOH solution and then to 8.5-9.5 with aqueous 10% KOH solution at an internal temperature of ≤ 20° C. The mixture was warmed to 20-25° C. and mixed for no less than 15 minutes. The lower aqueous solution was back extracted with dichloromethane (1045 g×2) at pH 8.5-9.5 (note: the pH value was decreased during the back extraction and additional 10% KOH aqueous solution may be needed to bring the pH value to the desired range of 8.5-9.5). The combined organic fractions were concentrated under vacuum and chased with tetrahydrofuran (700 g×2) to a final weight of 580 g. The solution of the title compound was diluted with tetrahydrofuran (700 g) and filtered through a layer of diatomaceous earth. The filter was rinsed with tetrahydrofuran (62 g). The combined filtrates were concentrated to a final weight of ˜ 427 g solution that is equal to 34.4 weight % title compound, assuming 100% yield for this reaction. The solution was directly used in the next step. However, an analytical sample was obtained by removal of the solvent to give the title compound, (3R)-3-amino-1-(morpholin-4-yl)-4-(phenylsulfanyl)butan-1-one (2-5). MS (ESI⁺) m/z 281.1 [M+H]⁺; ¹H NMR (400 MHZ, CD₃OD) δ ppm 7.40-7.45 (m, 2H), 7.27-7.34 (m, 2H), 7.21 (m, 1H), 3.59 (m, 4H), 3.52 (m, 2H), 3.43 (m, 2H), 3.28 (m, 1H), 3.14 (dd, 1H, J=13.7, 5.6 Hz), 2.97 (dd, 1H, J=13.7, 7.2 Hz), 2.67 (dd, 1H, J=16.1, 4.7 Hz), 2.44 (dd, 1H, J=16.1, 7.8 Hz).

### Example 12. (2R)-4-(morpholin-4-yl)-1-(phenylsulfanyl)butan-2-amine L-tartrate (2-6)

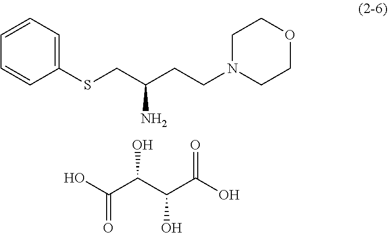

To a 5 L jacket flask equipped with a mechanical stirrer and temperature probe was charged sodium borohydride (98%, 52.8 g, 1.368 mole, 2.61 equivalents) and anhydrous tetrahydrofuran (850 g). The mixture was stirred and cooled down to 0-5° C. under N₂. The tetrahydrofuran solution of (3R)-3-amino-1-(morpholin-4-yl)-4-(phenylsulfanyl)butan-1-one (2-5) from above (427 g, 34.4 weight %, 0.524 mole) was added at an internal temperature of ≤ 10° C.) and rinsed with tetrahydrofuran (50 g). The internal temperature of the mixture was adjusted to 0-5° C. A solution of sulfuric acid (98%, 73.1 g, 0.729 mole, 1.39 equivalents) in tetrahydrofuran (320 g) was dosed slowly into the 5 L reactor under N₂ over ˜ 8 hours via a pump while maintaining the internal temperature 0-5° C. The resulting reaction mixture was warmed up slowly to 20-25° C. over 2 hours and stirred for 15 hours. The reaction mixture was cooled to 0-5° C., and 310 g of aqueous 15% HCl solution was added slowly at an internal temperature of ≤10° C. The quenched mixture was heated to 50-55° C. and stirred for 2 hours. The mixture was then concentrated under vacuum to a weight of approximately 970 g and chased with methanol (925 mL×2) and water (355 g) to a final weight of approximately 970 g. The mixture was diluted with water (355 g) and cooled down to 0-5° C. The pH of the mixture was adjusted to 6.0-7.0 with aqueous 20% KOH solution at an internal temperature of ≤20° C. Isopropyl acetate (555 g) was added, and the pH of the mixture was adjusted to 9.0-9.5 with aqueous 20% KOH solution at ≤20° C. The mixture was stirred at 20-25° C. for 15 minutes and then filtered through a layer diatomaceous earth. The filter was rinsed with isopropyl acetate (50 g). The filtered mixture was stirred for 15 minutes at 20-25° C. and settled for 30 minutes. The upper product phase was separated. The lower aqueous phase was back-extracted with isopropyl acetate (555 g) twice while maintaining the pH at 9.0-9.5 and an internal temperature at 20-25° C. The combined isopropyl acetate fractions were concentrated under vacuum to a weight of approximately 300 g at a jacket temperature of 40° C. and chased with isopropyl acetate (555 g) twice. The concentrated product solution was diluted with isopropyl acetate (555 g) and filtered. The filtrate was distilled under vacuum at the jacket temperature of 40° C. to a weight of ˜ 245 g. To a cleaned and dried reactor were charged diatomaceous earth (10 g) and n-heptane (196 g). The above product solution (˜245 g) was then added to a slurry of diatomaceous earth/n-heptane at 20-25° C. over 30 minutes and rinsed with isopropyl acetate (50 g). n-Heptane (980 g) was added slowly over 60-90 minutes. The mixture was stirred at 20-25° C. for 30 minutes and filtered through a filter aid. The filter was washed with n-heptane (176 g). The combined filtrates were concentrated to a weight of approximately 350 g under vacuum at a jacket temperature of 40° C. and chased with isopropyl acetate (960 g) three times. The weight of product solution was adjusted to a final weight of 524 g with isopropyl acetate. Methanol (402 g) and water (49 g) were added. The solution was adjusted to 20-25° C., and 138.0 g of 22.0% L-tartaric acid in methanol solution was added slowly over 1 hour. The solution was stirred at 20-25° C. for 1 hour. An additional 138.0 g of 22.0% L-tartaric acid in methanol was added slowly over 2 hours. The slurry was stirred at 20-25° C. for 1 hour and cooled to 0-5° C. over 2 hours. The slurry was mixed for 1 hour and collected by filtration. The wet cake was washed with 900 g of isopropyl acetate/methanol (1:1 by volume), and dried under vacuum at 60° C. for 12 hours. All isolated title compound was charged back to a reactor, followed by addition of methanol (560 g), isopropyl acetate (615 g) and water (212.5 g). The slurry was heated to 65° C. and mixed for 10 minutes or until all solids were dissolved. The solution was cooled down to 45-50° C., and 0.55 g of dried seed material of the title compound was added all at once. The seeds may be obtained from a disclosed synthesis, or they may be obtained from solids obtained by this procedure that were not seeded. The seeding is utilized to make the process more consistent. The solution was mixed at 45-50° C. for 1 hour and cooled down slowly to 30° C. over 6 hours. The resulting slurry was stirred at 30° C. for 1 hour, cooled down to 0-5° C. over 3 hours and stirred for 3 hours. The title compound, (2R)-4-(morpholin-4-yl)-1-(phenylsulfanyl)butan-2-amine L-tartrate (2-6), was collected by filtration and washed with 830 g of isopropyl acetate/methanol (1:1 by volume). The wet cake was then dried at 60° C. for 24 hours to give the title compound (135.0 g, 62% overall yield from benzyl [(2R)-1-hydroxy-4-(morpholin-4-yl)-4-oxobutan-2-yl]carbamate (2-2). mp 176° C. (from DSC); MS (ESI⁺) m/z 267.2 [M+H]⁺; 1H NMR (400 MHZ, DMSO-d₆) δ ppm 7.41 (m, 2H), 7.35 (m, 2H), 7.24 (m, 1H), 3.91 (s, 2H), 3.50 (m, 4H), 3.3-3.1 (m, 3H), 2.5-2.2 (m, 6H), 1.90-1.65 (m, 2H); ¹³C NMR (101 MHZ, DMSO-d₆/D₂O) δ ppm 175.58, 134.77, 130.17, 129.77, 127.50, 73.12, 66.39, 54.60, 53.23, 49.91, 35.57, 27.41.

### Example 13. 4-{[(2R)-4-(morpholin-4-yl)-1-(phenylsulfanyl)butan-2-yl]amino}-3-(trifluoromethanesulfonyl)benzene-1-sulfonamide (2-7)

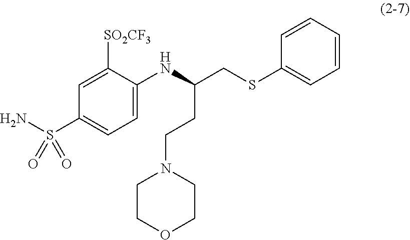

A reactor was charged with (2R)-4-(morpholin-4-yl)-1-(phenylsulfanyl)butan-2-amine L-tartrate (2-6) (1.61 kg), ethyl acetate (9.1 kg) and a solution comprised of potassium carbonate (2.5 kg) and water (7.5 kg). The mixture was mixed and permitted to settle, and the aqueous layer was separated. The organic phase was washed with a solution comprised of sodium chloride (2.5 kg) and water (7.5 kg). The organic phase was concentrated, diluted with acetonitrile (9.9 kg) and adjusted to a final volume of 7.1 L. A reactor was charged with 4-chloro-3-(trifluoromethanesulfonyl)benzene-1-sulfonamide (1-5) (basis charge), 1,4-diazabicyclo[2.2.2]octane (DABCO, 0.34 kg/kg), and the (2R)-4-(morpholin-4-yl)-1-(phenylsulfanyl)butan-2-amine (2-6) freebase solution. The reactor was sealed and heated to 95° C. for 24 hours. The reaction was cooled, ethyl acetate was charged (15.4 kg), and the organic solution was washed once with a solution comprised of potassium carbonate (0.9 kg) and water (7.8 kg), and twice with a solution comprised of sodium chloride (0.9 kg) and water (7.8 kg). The crude organic fraction was concentrated, diluted with ethyl acetate (28.0 kg) and adjusted to a final volume of 9.7 L. 2-Propanol (3.1 kg) was charged, and the solution was heated to 50° C., followed by the addition of n-heptane (11.1 kg). 4-{[(2R)-4-(Morpholin-4-yl)-1-(phenylsulfanyl)butan-2-yl]amino}-3-(trifluoromethanesulfonyl)benzene-1-sulfonamide (2-7) (90 g) was charged as seeds, followed by the addition of n-heptane (12.8 kg). The seeds may be obtained from a disclosed synthesis, or they may be obtained from solids obtained by this procedure that were not seeded. The seeding is utilized to make the process more consistent. The title compound, 4-{[(2R)-4-(morpholin-4-yl)-1-(phenylsulfanyl)butan-2-yl]amino}-3-(trifluoromethanesulfonyl)benzene-1-sulfonamide (2-7), was isolated via filtration, and the wet cake was washed with a solution comprised of ethyl acetate and n-heptane (32:68 v:v, 10.3 L). The wet cake was dried with heat and vacuum (50° C. and 100 mmHg), generating the title compound. HRMS (ESI) m/z calculated for C₂₁H₂₇F₃N₃O₅S₃⁺ [M+H]⁺ 554.10594: found 554.10277; ¹H NMR (700 MHZ, DMSO-d₆) δ ppm 7.98 (d, J=2.3 Hz, 1H), 7.85 (dd, J=9.3, 2.3 Hz, 1H), 7.37 (br s, 2H), 7.35 (m, 2H), 7.30 (m, 2H), 7.22 (m, 1H), 7.06 (d, J=9.5 Hz, 1H), 6.91 (d, J=9.1 Hz, 1H), 4.10 (m, 1H), 3.50 (m, 4H), 3.37 (dd, J=13.9, 5.2 Hz, 1H), 3.29 (dd, J=14.0, 7.2 Hz, 1H), 2.33 (br s, 2H), 2.31 (ddd, J=12.5, 8.6, 6.1 Hz, 1H), 2.26 (ddd, J=12.5, 6.2, 5.3 Hz, 1H), 2.17 (br m, 2H), 1.95 (dddd, J=14.6, 8.6, 6.4, 4.6 Hz, 1H), 1.73 (ddt, J=14.0, 8.1, 5.6 Hz, 1H); ¹³C NMR (176 MHz, DMSO-d₆) δ ppm 151.30, 135.51, 135.35, 131.18, 130.99, 129.07, 128.96, 126.19, 119.77 (q, J=326.6 Hz), 114.61, 105.67, 66.08, 53.91, 53.14, 50.40, 37.17, 29.84.

### Example 14. 2-chloro-5,5-dimethylcyclohex-1-ene-1-carbaldehyde (3-2)

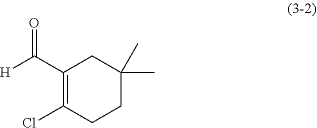

To a 100 mL Erlenmeyer flask were charged 4,4-dimethylcyclohexanone (3-1) (505.2 g, 4.0 mol) and dichloromethane (400.2 g), and the contents were mixed to dissolve solids. A 3 L jacketed flask was charged with anhydrous N,N-dimethylformamide (452.2 g, 6.2 mol, 1.55 equivalents) and dichloromethane (1557.0 g). The content was cooled to 0±5° C., and POCI₃ (921.0 g, 6.0 mol, 1.50 equivalents) was slowly added over a period of 1 hour, and residual POCI₃ was rinsed into the reaction mixture with dichloromethane (133.4 g). The reaction mixture temperature was slowly adjusted to 20±5° C. and mixed for 1 hour. The 4,4-dimethylcyclohexanone (3-1) in dichloromethane solution was charged to the reaction mixture over a period of approximately 4 hours maintaining the temperature at 20±5° C. The Erlenmeyer flask was rinsed with dichloromethane (268.50 g), and the rinse was charged to the reaction mixture. The reaction mixture was stirred at 20±5° C. for 5 hours and heated to 40±2° C. for no less than 13 hours.

To a 10 L jacketed flask provided with a mechanical stirrer were charged sodium acetate (327.9 g, 3.98 mol, 1 equivalent), sodium chloride (234.5 g) and water (2555.0 g). The mixture was stirred to dissolve solids. Dichloromethane (873.9 g) was charged to the aqueous salt solution, and the mixture was cooled to 0±5° C. The above reaction mixture was slowly quenched into the biphasic mixture over a period of 1 hour maintaining a temperature of 0±5° C. The quenched reaction mixture was adjusted to 20±5° C. and stirred for no less than 30 minutes and allowed to settle for no less than 15 minutes. The lower organic phase was separated. The upper aqueous phase was back extracted with dichloromethane (875.6 g).

To a 10 L Erlenmeyer flask provided with a magnetic stirrer were charged K₃PO₄ (173.5 g, 0.82 mol), sodium chloride (468.5 g) and water (7172.4 g). The mixture was stirred to dissolve solids.

The 2.2 weight % K₃PO₄ solution (3907.2 g) was charged to the dichloromethane reaction mixture, and the resultant mixture was stirred at 25±5° C. for 15 minutes and then allowed to settle for 15 minutes. The lower organic layer was separated. Another aliquot of 2.2 weight % K₃PO₄ solution (3907.2 g) was charged to the lower organic layer, and the mixture was stirred at 25±5° C. for 15 minutes and allowed to settle for 15 minutes. The lower organic layer was separated and concentrated under reduced pressure at no more than 40° C. The residue was subjected to constant volume distillation with acetonitrile (4966.5 g). The resulting title compound, 2-chloro-5,5-dimethylcyclohex-1-ene-1-carbaldehyde (3-2), was carried to next step as an acetonitrile solution assuming quantitative conversion. MS (CI) m/z 171.06 [M−H]; ¹H NMR (400 MHZ, DMSO-d₆) δ ppm 10.09 (s, H), 2.61 (m, 2H), 1.99 (br, 2H), 1.49 (t, J=6.5 Hz, 2H), 0.90 (s, 6H); ¹³CNMR (101 MHz, DMSO-d₆) δ ppm 190.66, 149.90, 131.60, 36.71, 34.99, 33.20, 28.01, 27.23.

### Example 15. 4′-chloro-4,4-dimethyl-3,4,5,6-tetrahydro-[1,1′-biphenyl]-2-carbaldehyde (3-3)

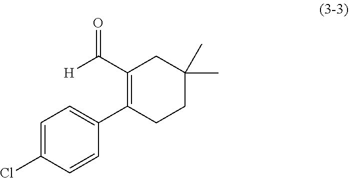 [SMILES: CC1(C)CCC(c2ccc(Cl)cc2)=C(C=O)C1]

To a 5 L media bottle provided with a mechanical stirrer were charged potassium phosphate dibasic (1864.4 g) and water (3803.0 g). The content was mixed to dissolve solids.

To a 10 L jacketed reactor were charged 2-chloro-5,5-dimethylcyclohex-1-ene-1-carbaldehyde solution (3-2) (691.2 g, 4.0 mol), tetrabutylammonium bromide (1244.3 g, 3.86), 4-chlorophenylboronic acid (603.7 g, 3.86 mol), and aqueous potassium phosphate dibasic solution. The mixture was stirred at 25±5° C. to dissolve solids (biphasic system). The mixture was evacuated and purged with N₂ three times. Palladium acetate (2.08 mg, 9.3 mmol) was added all at once under N₂. The reaction mixture was evacuated and purged with N₂ three times. The reaction mixture was heated to 65±5 C for 18 hours. Then the reaction mixture was cooled to 25±5° C. and allowed to settle. The aqueous layer was separated. The organic layer was filtered through a filter aid to remove solids and transferred to a clean 10 L jacketed reactor. The original reactor was rinsed with toluene (3512.4 g), and the filtered rinse was added to the reactor containing the filtered reaction mixture. The reaction was slowly quenched with 12 weight % NaOH solution (2272.6 g) maintaining the temperature at 25±5° C. The suspension was stirred for 30 minutes and allowed to settle for 15 minutes. The lower aqueous layer was discarded.

To a 2 L media bottle provided with a magnetic stirrer were charged sodium bicarbonate (101.72 g), L-cysteine (40.12 g) and water (1919.6 g). The mixture was stirred to dissolve solids. The resultant L-cysteine solution was charged to the 10 L reactor above at 25±5° C., stirred for 30 minutes and allowed to settle for 15 minutes. The lower aqueous layer was separated. The upper organic layer was concentrated at no more than 40° C. under reduced pressure, subjected to constant volume distillation with toluene (7620 g) to obtain 2531 g (˜ 995.8 g title compound) of the title compound, 4′-chloro-4,4-dimethyl-3,4,5,6-tetrahydro-[1,1′-biphenyl]-2-carbaldehyde (3-3), mixture used without additional purification. MS (CI) m/z 247.09 [M−H]⁻; ¹H NMR (400 MHZ, DMSO-d₆) δ ppm 9.38 (s, 1H), 7.47 (d, J=8.4 Hz, 7.37 (d, J=8.4 Hz, 2H), 2.54 (m, 2H), 2.03 (br, 2H), 1.48 (t, J=6.4 Hz, 2H), 0.96 (s, 6H); ¹³C NMR (101 MHz, DMSO-d₆) δ ppm 192.12, 156.60, 137.52, 134.02, 133.13, 130.49, 128.26, 35.59, 34.40, 31.11, 27.84, 27.66.

### Example 16. tert-butyl 4-[(4′-chloro-4,4-dimethyl-3,4,5,6-tetrahydro[1,1′-biphenyl]-2-yl)methyl]piperazine-1-carboxylate (3-4)

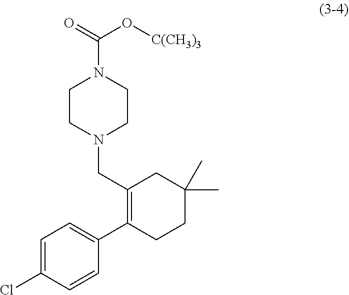 [SMILES: CC1(C)CCC(c2ccc(Cl)cc2)=C(CN2CCN(C(=O)OC(C)(C)C)CC2)C1]

To a 10 L three-neck jacketed flask provided with a mechanical stirrer were charged a solution of 4′-chloro-4,4-dimethyl-3,4,5,6-tetrahydro-[1,1′-biphenyl]-2-carbaldehyde (3-3) (2530.9 g, ˜4.0 mol) in toluene (1888 g), tert-butyl piperazine-1-carboxylate (697.5 g, 3.75 mol) and anhydrous tetrahydrofuran (3310 g). The solution was stirred at 25±5° C. for 5 minutes. Sodium triacetoxyborohydride (762.70 g, 3.6 mol) was added in portion over a period of two hours maintaining the reaction temperature at 25±5° C. The mixture was stirred at 25±5° C. for no less than 12 hours.

Citric acid solution (10 weight %, 3734.5 g) was charged to the reaction mixture, and the mixture was stirred at 25±5° C. for 30 minutes and allowed to settle for 15 minutes. The lower aqueous layer was separated. Citric acid solution (12 weight %, 3734.0 g) was charged to the upper product layer, and the mixture was stirred at 25±5° C. for 30 minutes and allowed to settle for 15 minutes. The lower aqueous layer was separated. Sodium bicarbonate solution (5 weight %, 3265.2 g) was charged to the upper product layer, and the mixture was stirred at 25±5° C. for 30 minutes and allowed to settle for 15 minutes. The lower aqueous layer was separated. Sodium bicarbonate solution (5 weight %, 3265.9 g) was charged to the upper product layer, the mixture was stirred at 25±5° C. for 30 minutes and allowed to settle for 15 minutes. The lower aqueous layer was separated. Sodium chloride solution (25 weight %, 3464.0 g) was charged to the upper product layer, and the mixture was stirred at 25±5° C. for 30 minutes and allowed to settle for 15 minutes. The lower aqueous layer was separated. The separated organic layer was concentrated at 45±5° C. under reduced pressure, toluene (4725 g) was added to the residue, and the mixture was concentrated to dryness. The residue was dissolved in acetonitrile (4816.0 g) at 80±5° C., and slowly cooled to −10±2° C. over a period of 10 hours and then mixed for 2 hours. The title compound was collected by filtration and rinsed with pre-cooled acetonitrile (2165.0 g). The solid tert-butyl 4-[(4′-chloro-4,4-dimethyl-3,4,5,6-tetrahydro[1,1′-biphenyl]-2-yl)methyl]piperazine-1-carboxylate (3-4) was dried under vacuum at 50° C. overnight (1079.1 g, 64%). MS (CI) m/z 419.25 [M+H]⁺; 1H NMR (400 MHZ, CDCl₃) § ppm 7.29 (d, J=8.3 Hz, 2H), 6.99 (d, J=8.3 Hz, 2H), 3.36 (t 5.0 Hz, 4H), 2.74 (s, 2H), 2.24 (t, J=6.3 Hz, 2H), 2.15 (t 5.1 Hz, 4H), 2.00 (s, 2H), 1.47 (t, J=6.4 Hz, 2H), 1.45 (3H), 1.45 (s, 9H), 0.99 (s, 6H); ¹³C NMR (101 MHz, CDCl₃) δ ppm 154.70, 141.93, 134.46, 131.94, 129.58, 128.23, 79.42, 60.68, 52.55, 43.28, 41.39, 35.64, 30.82, 28.92, 28.41, 28.11.

### Example 17. 1-[(4′-chloro-4,4-dimethyl-3,4,5,6-tetrahydro[1,1′-biphenyl]-2-yl)methyl]piperazine (3-5)

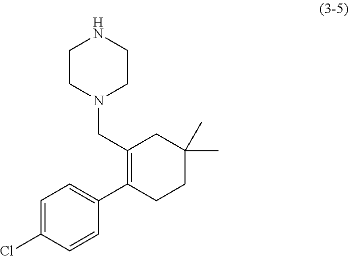 [SMILES: CC1(C)CCC(c2ccc(Cl)cc2)=C(CN2CCNCC2)C1]

To a 10 L three-neck round bottom flask equipped with a mechanical stirrer were charged tert-butyl 4-[(4′-chloro-4,4-dimethyl-3,4,5,6-tetrahydro[1,1′-biphenyl]-2-yl)methyl]piperazine-1-carboxylate (3-4) (1074.1 g, 2.56 mol) and isopropyl alcohol (8377.5 g). The mixture was stirred at ambient temperature for 5 minutes and then concentrated HCl (880.8 g) was added to the slurry. The reaction mixture was adjusted to an internal temperature of 65±5° C. The reaction mixture was agitated at 65±5° C. for no less than 12 hours.

The product slurry was cooled down to −5° C. slowly (10° C./hour). The product slurry was mixed at near −5° C. for no less than 2 hours, and the solids were collected by filtration. The wet cake was washed with isopropyl alcohol (72 mL) and dried at 50° C. under vacuum overnight to give 1047.1 g (90.4%) of the title compound 1-[(4′-chloro-4,4-dimethyl-3,4,5,6-tetrahydro[1,1′-biphenyl]-2-yl)methyl]piperazine (3-5) as a bis-hydrochloride isopropyl alcohol solvate (purity 99.4% peak area at 220 nm). MS (CI) m/z 319.19 [M+H]⁺; ¹H NMR (400 MHz, DMSO-d₆) δ ppm 11.51 (br, 1H), 9.94 (s, 1H), 9.59 (s, 1H), 7.43 (d, J=8.4 Hz, 2H), 7.19 (d, J=8.5 Hz, 2H), 3.51 (br, 2H), 3.51 (br, 2H), 3.49 ((br, 2H), 3.33 (br, 2H), 2.90 (br, 2H), 2.25 (t, J=6.0 Hz, 2H), 2.21 (br, 2H), 1.44 (t, J=6.4 Hz, 2H), 0.97 (s, 6H); ¹³C NMR (101 MHZ, DMSO-d₆) & ppm 141.77, 140.12, 131.84, 129.87, 128.66, 121.92, 58.69, 47.65, 41.23, 39.46, 34.55, 30.84, 28.70, 27.71.

### Example 18. ethyl 4-{4-[(4′-chloro-4,4-dimethyl-3,4,5,6-tetrahydro[1,1′-biphenyl]-2-yl)methyl]piperazin-1-yl}benzoate (3-6)

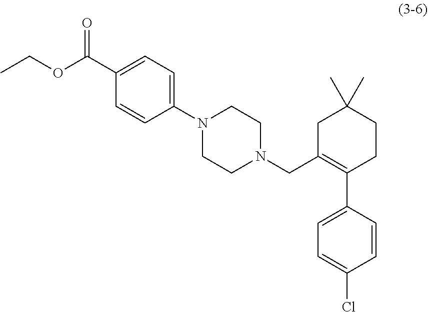 [SMILES: CCOC(=O)c1ccc(N2CCN(CC3=C(c4ccc(Cl)cc4)CCC(C)(C)C3)CC2)cc1]

A 50 mL flask with a magnetic stir bar was charged with sulfolane (25 mL), 1-[(4′-chloro-4,4-dimethyl-3,4,5,6-tetrahydro[1,1′-biphenyl]-2-yl)methyl]piperazine (3-5) (5.0 g, 11.06 mmol), 1,8-diazabicyclo[5.4.0]undec-7-ene (5.05 g, 33.20 mmol, 3.0 equivalents) and ethyl 4-fluorobenzoate (5.58 g, 33.20 mmol, 3.0 equivalents). The contents of the flask were stirred at 120° C. for 12 hours. After this time, the reaction mixture was allowed to cool at 95° C. and seeded with the title compound (0.005 g, 0.01 mmol). The seeds may be obtained from a disclosed synthesis, or they may be obtained from solids obtained by this procedure that were not seeded. The seeding is utilized to make the process more consistent. The slurry was allowed to cool at 50° C. and methanol (60 mL) was charged. The slurry was allowed to cool to 25° C. and then the solids were isolated by filtration. The wet cake was rinsed with methanol (30 mL). The solids were dried at 50° C. under vacuum to afford the title compound, ethyl 4-{4-[(4′-chloro-4,4-dimethyl-3,4,5,6-tetrahydro[1,1′-biphenyl]-2-yl)methyl]piperazin-1-yl}benzoate (3-6), (4.56 g, 9.76 mmol, 88%) with a purity of 99.80%. MS (CI) m/z 467.25 [M+H]⁺; ¹H NMR (400 MHZ, CDCl₃) δ ppm 7.91 (d, J=8.9 Hz, 2H), 7.28 (d, J=8.4 Hz, 2H), 7.01 (d, J=8.3 Hz, 2H), 6.82 (d, J=8.9 Hz, 2H), 4.33 (q, J=7.1 Hz, 2H), 3.26 (t, J=5.1 Hz, 2H), 2.80 (s, 2H), 2.36 (t, J=5.1 Hz, 2H), 2.26 (t, J=6.5 Hz, 2H), 2.03 (s, 2H), 1.47 (t, J=6.5 Hz, 2H), 1.36 (d, J=7.1 Hz, 3H), 1.00 (s, 6H); ¹³C NMR (101 MHz, CDCl₃) δ ppm 166.90, 154.38, 142.12, 134.81, 132.20, 131.29, 129.94, 129.76, 128.46, 120.03, 113.68, 60.83, 60.49, 52.66, 47.76, 41.67, 35.86, 31.05, 29.17, 28.34, 14.65.

### Example 19. 4-{4-[(4′-chloro-4,4-dimethyl-3,4,5,6-tetrahydro[1,1′-biphenyl]-2-yl)methyl]piperazin-1-yl}benzoic acid (3-7)

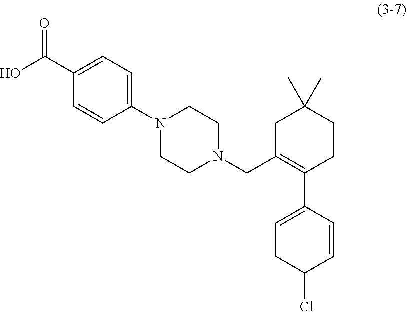 [SMILES: CC1(C)CCC(C2=CCC(Cl)C=C2)=C(CN2CCN(c3ccc(C(=O)O)cc3)CC2)C1]

To a 300 mL jacketed flask equipped with a mechanical stirrer were charged ethyl 4-{4-[(4′-chloro-4,4-dimethyl-3,4,5,6-tetrahydro[1,1′-biphenyl]-2-yl)methyl]piperazin-1-yl}benzoate (3-6) (20.0 g, 42.9 mmol), ethanol (63.1 g), NaOH (8.6 g, 0.21 mol) and water (20.0 g). The reaction mixture was adjusted to an internal temperature of 75±5° C. The reaction mixture was agitated at 75±5° C. for no less than 2 hours.

The reaction mixture was diluted with water (160 g), and the pH was adjusted to 7.4 to 7.7 with orthophosphoric acid (11.3 g, H₃PO₄, 85 weight %) maintaining the reaction mixture temperature at 75±5° C. The mixture was seeded with the title compound (0.010 g, 0.021 mmol) and mixed for two hours. The seeds may be obtained from a disclosed synthesis, or they may be obtained from solids obtained by this procedure that were not seeded. The seeding is utilized to make the process more consistent. The pH was adjusted to 6.6 to 7.3 with orthophosphoric acid (3.6 g, H₃PO₄, 85 weight %) maintaining temperature at 75±5° C. and mixed for one hour. The resulting slurry was diluted with ethanol (34.8 g) and mixed at 75±5° C. for 30 minutes. The mixture was cooled to 50±5° C. and mixed for one hour. The slurry was diluted over a period of one hour with water (86.3 g) maintaining a temperature of 50±5° C. The slurry was cooled to 25±5° C., and the pH was adjusted to 6.1 to 6.4 with orthophosphoric acid (5.6 g, H₃PO₄) while maintaining the temperature at 25±5° C.

The slurry was mixed at 25±5° C. for no less than 1 hour, and the solids were collected by filtration. The wet cake was washed with 25 weight % aqueous ethanol (234.8 g), water (200 g) and ethanol (15.8 g) and dried at 50° C. under vacuum overnight to give 18.25 g (97.1%) of the title compound, 4-{4-[(4′-chloro-4,4-dimethyl-3,4,5,6-tetrahydro[1,1′-biphenyl]-2-yl)methyl]piperazin-1-yl}benzoic acid (3-7), as a zwitterion (purity >100.0% peak area at 210 nm). MS (CI) m/z 439.21 [M+H]⁺; ¹H NMR (400 MHZ, DMSO-d₆) δ ppm 12.26 (br, 1H), 7.75 (d, J=8.6 Hz, 2H), 7.37 (d, J=8.2 Hz, 2H), 7.09 (d, J=8.1 Hz, 2H), 6.90 (d, J=8.7 Hz, 2H), 3.21 (br, 4H), 2.73 (s, 2H), 2.25 (br, 4H), 2.21 (br, 2H), 1.98 (s, 2H), 1.41 (t, J=6.4 Hz, 2H), 0.95 (s, 6H); ¹³C NMR (101 MHz, DMSO-d₆) δ ppm 167.23, 153.64, 141.77, 133.60, 130.88, 130.79, 130.04, 129.27, 128.06, 119.34, 113.21, 59.92, 52.14, 46.80, 41.01, 35.14, 30.20, 28.55, 27.95.

### Example 20. 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (3-8)

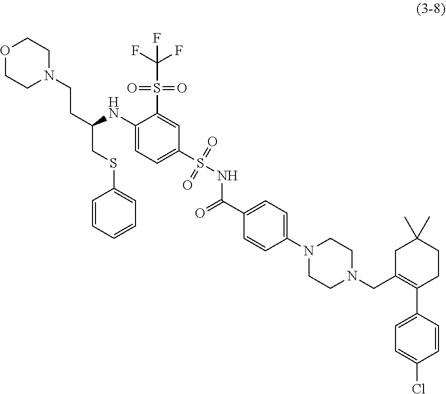 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

To a mixture of 4-{[(2R)-4-(morpholin-4-yl)-1-(phenylsulfanyl)butan-2-yl]amino}-3-(trifluoromethanesulfonyl)benzene-1-sulfonamide (2-7) (12.0 g, 21.7 mmol), 4-{4-[(4′-chloro-4,4-dimethyl-3,4,5,6-tetrahydro[1,1′-biphenyl]-2-yl)methyl]piperazin-1-yl}benzoic acid (3-7) (10.5 g, 23.8 mmol), N,N-dimethylpyridin-4-amine (3.2 g, 26.0 mmol) and 1-ethyl-3-[3-(dimethylamino)propyl]-carbodiimide hydrochloride (4.8 g, 24.9 mmol) was added ethyl acetate (60 mL, 5 mL/g) and triethylamine (2.2 g, 21.7 mmol). The reaction mixture was heated to 45° C. and monitored by HPLC for reaction conversion. See Table 5 for the HPLC conditions. The reaction was quenched with N,N-dimethylethane-1,2-diamine (1.9 g, 21.7 mmol) and stirred at 45° C. for 1 hour. The mixture was cooled to 25° C., diluted with ethyl acetate (84 mL, 7 mL/g), and extracted with 10% acetic acid+0.8% sodium chloride (120 mL, 2×). The pH was adjusted to pH 5-7 using 3-5% sodium bicarbonate. The crude solution was concentrated to approximately 49 g (1.6 mL/g). The solution was heated to 42° C. and ethanol (115 g, 80:20 vs ethyl acetate) was added. The mixture was seeded (1%) and held at 42° C. for 5 hours for the initial precipitation. The seeds may be obtained from a disclosed synthesis, or they may be obtained from solids obtained by this procedure that were not seeded. The seeding is utilized to make the process more consistent. Then more ethanol (144 g, dilute to 90:10 vs ethyl acetate) was added at 42° C., and then the mixture was cooled to 2° C. The title compound was collected by filtration, washed with 5% ethyl acetate in ethanol solution, and dried under vacuum with heat (no more than 90° C.) to give the title compound 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (3-8). HRMS (ESI) m/z calculated for C₄₇H₅₆ClF₃N₅O₆S₃⁺ [M+H]⁺ 974.30279: measured 974.30355; ¹H NMR (400 MHZ, CDCl₃) δ ppm 8.13 (d, J=2.2 Hz, 1H), 7.95 (dd, J=9.1, 2.2 Hz, 1H), 7.72 (d, J=9.1 Hz, 2H), 7.37 (d, J=8.4 Hz, 2H), 7.32 (dd, J=8.3, 1.3 Hz, 2H), 7.26 (t, J=7.6 Hz 2H), 7.17 (tt, J=7.3, 1.3 Hz, 1H), 7.11 (d, J=8.4 Hz, 2H), 6.99 (d, J=9.4 Hz, 1H), 6.88 (d, J=8.9 Hz, NH), 6.88 (d, J=9.2 Hz, 2H), 4.07 (m, 1H), 3.54 (br, 2H), 3.35 (dd, J=13.9, 5.2 Hz, 2H), 3.23 (br, 2H), 2.86 (s, 2H), 2.51 (m, 6H), 2.39 (br, 4H), 2.22 (t, J=6.1 Hz, 2H), 1.98 (m, 2H), 1.71 (s, 2H), 1.42 (t, J=6.5 Hz, 2H), 0.96 (s, 6H); ¹³C NMR (176 MHZ, DMSO-d₆) δ ppm 166.8, 152.8, 151.1, 141.5, 137.1, 135.2, 134.8, 133.5, 131.0, 130.0, 129.8, 129.0, 128.2, 126.2, 123.5, 113.7, 113.1, 105.8, 65.4, 59.7, 53.7, 52.7, 51.9, 50.4, 46.5, 40.9, 37.1, 35.0, 30.3, 29.2, 28.6, 27.9.

TABLE 5
HPLC conditions:

| Column | Waters Xbridge BEH C18, 100 × 4.6 mm, 2.5 μm | | |
|---|---|---|---|
| Column Temp | 45° C. | | |
| Wavelength | 220 nm | | |
| Sample Tray | ambient | | |
| Flow rate | 1.5 mL/minute | | |
| Injection Volume | 5 mL | | |
| Mobile Phase | A: 0.1% perchloric acid in water (v/v) | | |
| | B: acetonitrile | | |
| Gradient Table | Time (minutes) | % A | % B |
| | 0 | 68 | 32 |
| | 1.7 | 68 | 32 |
| | 10.7 | 38 | 62 |
| | 12.7 | 10 | 90 |
| | 14.7 | 10 | 90 |
| | 14.8 | 68 | 32 |
| | 19.0 | 68 | 32 |

| | |
|---|---|
| Run Time | 19 minutes |
| Sample Diluent | acetonitrile |

All references cited herein are incorporated by reference in their entirety. While the methods provided herein have been described with respect to the particular embodiments, it will be apparent to those skilled in the art that various changes and modifications can be made without departing from the spirit and scope as recited by the appended claims.

The embodiments described above are intended merely to be exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method for preparing 1-chloro-2-[(trifluoromethyl)sulfanyl]benzene having structure (1-2): 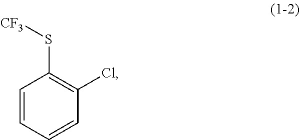 [SMILES: FC(F)(F)Sc1ccccc1Cl]
the method comprising:
alkylating 2-chlorobenzene-1-thiol having structure (1-1): 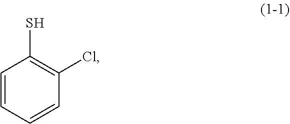
with trifluoroiodomethane (CF₃I) to thereby prepare 1-chloro-2-[(trifluoromethyl)sulfanyl]benzene having structure (1-2).

2. The method of claim 1, wherein alkylating 2-chlorobenzene-1-thiol having structure (1-1) is catalyzed by irradiation.

3. The method of claim 2, wherein the mixture is irradiated with visible light.

4. The method of claim 1, wherein alkylating 2-chlorobenzene-1-thiol having structure (1-1) is catalyzed by irradiation in the presence of a photoredox catalyst.

5. The method of claim 4, wherein the photoredox catalyst is tris(2,2′-bipyridyl)dichlororuthenium(II) hexahydrate (Ru(bpy)₃Cl₂(H₂O)₆).

6. The method of claim 1, wherein alkylating 2-chlorobenzene-1-thiol having structure (1-1) occurs in a reaction mixture prepared by combining a first feed solution comprising 2-chlorobenzene-1-thiol having structure (1-1), 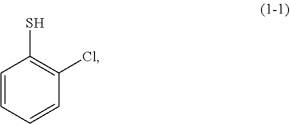
and a second feed solution comprising trifluoroiodomethane.

7. The method of claim 6, wherein the first feed solution further comprises a photoredox catalyst.

8. The method of claim 6, wherein the second feed solution further comprises an amine base.

9. The method of claim 6, wherein the reaction mixture is irradiated with visible light.

10. A method for preparing 1-chloro-2-(trifluoromethylsulfonyl)benzene having structure (1-3): 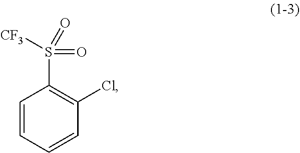 [SMILES: O=S(=O)(c1ccccc1Cl)C(F)(F)F]
the method comprising:
contacting 1-chloro-2-[(trifluoromethyl)sulfanyl]benzene having structure (1-2): 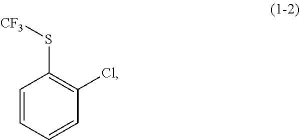 [SMILES: FC(F)(F)Sc1ccccc1Cl]
with an oxidizing agent to thereby prepare 1-chloro-2-(trifluoromethylsulfonyl)benzene having structure (1-3).

11. The method of claim 10, wherein 1-chloro-2-[(trifluoromethyl)sulfanyl]benzene having structure (1-2) is prepared by alkylating 2-chlorobenzene-1-thiol having structure (1-1): 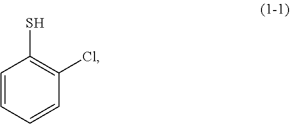
with trifluoroiodomethane to thereby prepare 1-chloro-2-[(trifluoromethyl)sulfanyl]benzene having structure (1-2).

12. The method of claim 11, wherein the method is continuous from the preparation of 1-chloro-2-[(trifluoromethyl)sulfanyl]benzene having structure (1-2) from 2-chlorobenzene-1-thiol having structure (1-1), and the preparation of 1-chloro-2-((trifluoromethyl)sulfonyl)benzene having structure (1-3) from 1-chloro-2-[(trifluoromethyl)sulfanyl]benzene having structure (1-2).

13. A method for preparing (2R)-4-(morpholin-4-yl)-1-(phenylsulfanyl)butan-2-amine L-tartrate having structure (2-6): 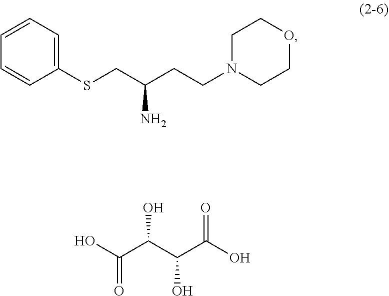 [SMILES: N[C@H](CCN1CCOCC1)CSc1ccccc1 ; O=C(O)[C@H](O)[C@@H](O)C(=O)O]
the method comprising:
contacting (3R)-3-amino-1-(morpholin-4-yl)-4-(phenylsulfanyl)butan-1-one having structure (2-5): 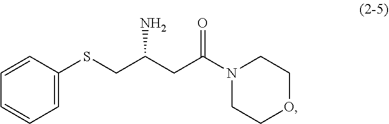 [SMILES: N[C@@H](CSc1ccccc1)CC(=O)N1CCOCC1]
with a borohydride and acid to thereby prepare (2R)-4-(morpholin-4-yl)-1-(phenylsulfanyl)butan-2-amine;
contacting (2R)-4-(morpholin-4-yl)-1-(phenylsulfanyl)butan-2-amine with L-tartaric acid to thereby prepare (2R)-4-(morpholin-4-yl)-1-(phenylsulfanyl)butan-2-amine L-tartrate having structure (2-6).

14. A method for preparing 4-{[(2R)-4-(morpholin-4-yl)-1-(phenylsulfanyl)butan-2-yl]amino}-3-(trifluoromethanesulfonyl)benzene-1-sulfonamide having structure (2-7): 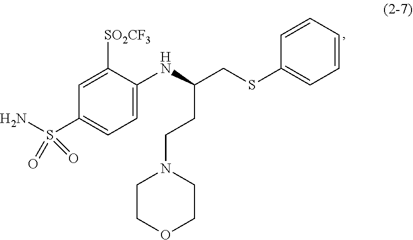
the method comprising:
converting (2R)-4-(morpholin-4-yl)-1-(phenylsulfanyl)butan-2-amine L-tartrate having structure (2-6): 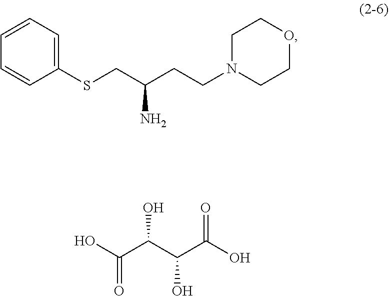 [SMILES: N[C@H](CCN1CCOCC1)CSc1ccccc1 ; O=C(O)[C@H](O)[C@@H](O)C(=O)O]
into free base (2R)-4-(morpholin-4-yl)-1-(phenylsulfanyl)butan-2-amine; and
coupling free base (2R)-4-(morpholin-4-yl)-1-(phenylsulfanyl)butan-2-amine with 4-chloro-3-(trifluoromethanesulfonyl)benzene-1-sulfonamide having structure (1-5): 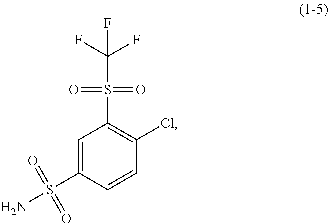 [SMILES: NS(=O)(=O)c1ccc(Cl)c(S(=O)(=O)C(F)(F)F)c1]
in an aprotic solvent catalyzed by a base to thereby prepare 4-{[(2R)-4-(morpholin-4-yl)-1-(phenylsulfanyl)butan-2-yl]amino}-3-(trifluoromethanesulfonyl)benzene-1-sulfonamide having structure (2-7).

15. A method for preparing ethyl 4-{4-[(4′-chloro-4,4-dimethyl-3,4,5,6-tetrahydro[1,1′-biphenyl]-2-yl)methyl]piperazin-1-yl}benzoate having structure (3-6): 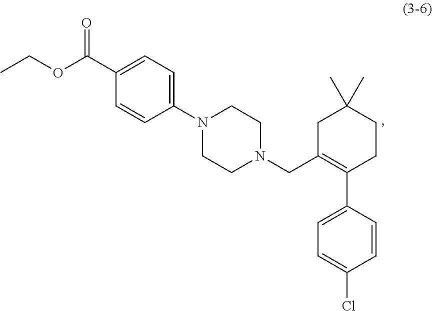 [SMILES: CCOC(=O)c1ccc(N2CCN(CC3=C(c4ccc(Cl)cc4)CCC(C)(C)C3)CC2)cc1]
the method comprising:
reacting 1-[(4′-chloro-4,4-dimethyl-3,4,5,6-tetrahydro[1,1′-biphenyl]-2-yl)methyl]piperazine having structure (3-5): 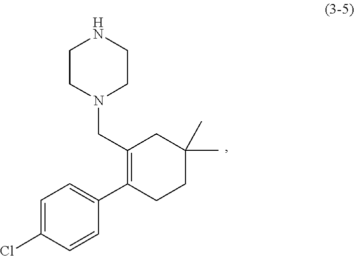
with ethyl 4-fluorobenzoate in the presence of an amine base in an aprotic solvent to thereby prepare ethyl 4-{4-[(4′-chloro-4,4-dimethyl-3,4,5,6-tetrahydro[1,1′-biphenyl]-2-yl)methyl]piperazin-1-yl}benzoate having structure (3-6).

16. A method for preparing 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (ABT-263) having structure (3-8): 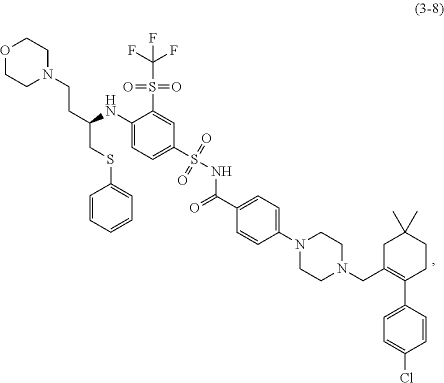 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
the method comprising coupling 4-{4-[(4′-chloro-4,4-dimethyl-3,4,5,6-tetrahydro[1,1′-biphenyl]-2-yl)methyl]piperazin-1-yl}benzoic acid having structure (3-7): 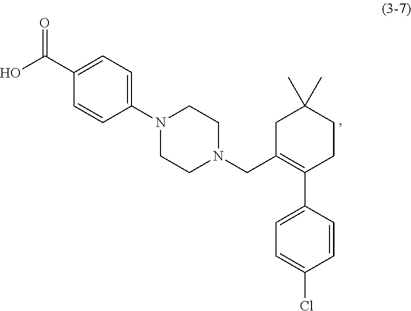 [SMILES: CC1(C)CCC(c2ccc(Cl)cc2)=C(CN2CCN(c3ccc(C(=O)O)cc3)CC2)C1]
with 4-{[(2R)-4-(morpholin-4-yl)-1-(phenylsulfanyl)butan-2-yl]amino}-3-(trifluoromethanesulfonyl)benzene-1-sulfonamide having structure (2-7): 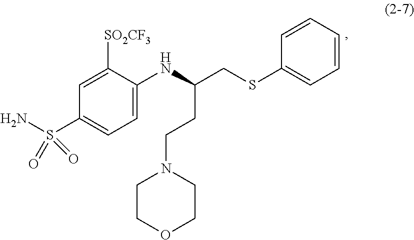
to thereby prepare 4-{4-[(4′-chloro-4,4-dimethyl-3,4,5,6-tetrahydro[1,1′-biphenyl]-2-yl)methyl]piperazin-1-yl}-N-[4-{[(2R)-4-(morpholin-4-yl)-1-(phenylsulfanyl)butan-2-yl]amino}-3-(trifluoromethanesulfonyl)benzene-1-sulfonyl]benzamide (ABT-263) having structure (3-8);
wherein 4-{[(2R)-4-(morpholin-4-yl)-1-(phenylsulfanyl)butan-2-yl]amino}-3-(trifluoromethanesulfonyl)benzene-1-sulfonamide (2-7) is prepared by coupling a free base of (2R)-4-(morpholin-4-yl)-1-(phenylsulfanyl)butan-2-amine with 4-chloro-3-(trifluoromethanesulfonyl)benzene-1-sulfonamide having structure (1-5): 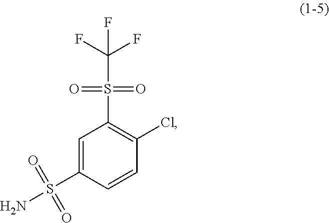 [SMILES: NS(=O)(=O)c1ccc(Cl)c(S(=O)(=O)C(F)(F)F)c1]
in an aprotic solvent catalyzed by a base.

17. The method of claim 16, wherein 4-{4-[(4′-chloro-4,4-dimethyl-3,4,5,6-tetrahydro[1,1′-biphenyl]-2-yl)methyl]piperazin-1-yl}benzoic acid having structure (3-7): 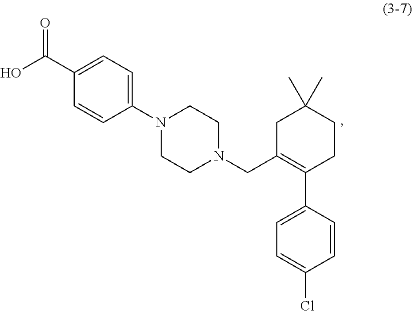 [SMILES: CC1(C)CCC(c2ccc(Cl)cc2)=C(CN2CCN(c3ccc(C(=O)O)cc3)CC2)C1]
is prepared by hydrolyzing ethyl 4-{4-[(4′-chloro-4,4-dimethyl-3,4,5,6-tetrahydro[1,1′-biphenyl]-2-yl)methyl]piperazin-1-yl}benzoate having structure (3-6): 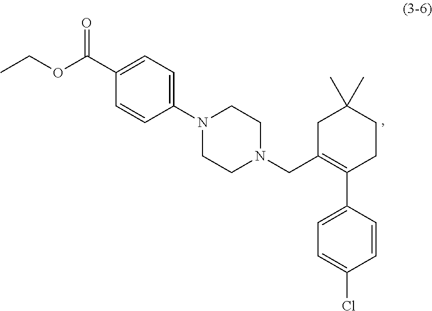 [SMILES: CCOC(=O)c1ccc(N2CCN(CC3=C(c4ccc(Cl)cc4)CCC(C)(C)C3)CC2)cc1]
with a hydroxide base in a solvent composition comprising ethanol and water.

18. The method of claim 17, wherein ethyl 4-{4-[(4′-chloro-4,4-dimethyl-3,4,5,6-tetrahydro[1,1′-biphenyl]-2-yl)methyl]piperazin-1-yl}benzoate having structure (3-6) is prepared by reacting 1-[(4′-chloro-4,4-dimethyl-3,4,5,6-tetrahydro[1,1′-biphenyl]-2-yl)methyl]piperazine having structure (3-5): 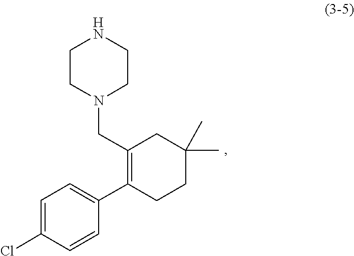
with ethyl 4-fluorobenzoate in the presence of an amine base in an aprotic solvent.

19. The method of claim 16, wherein 4-chloro-3-(trifluoromethanesulfonyl)benzene-1-sulfonamide having structure (1-5) is prepared by reacting 4-chloro-3-(trifluoromethanesulfonyl)benzene-1-sulfonyl chloride having structure (1-4): 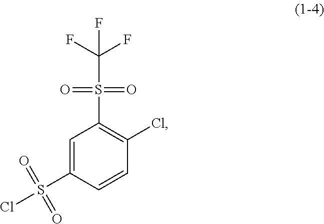 [SMILES: O=S(=O)(Cl)c1ccc(Cl)c(S(=O)(=O)C(F)(F)F)c1]
with aqueous ammonia.

20. The method of claim 19, wherein 4-chloro-3-(trifluoromethanesulfonyl)benzene-1-sulfonyl chloride having structure (1-4) is prepared by:
(a) irradiating a mixture comprising 2-chlorobenzene-1-thiol having structure (1-1), 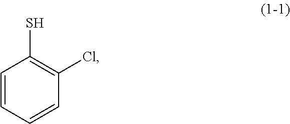
a photoredox catalyst, and trifluoroiodomethane to thereby prepare 1-chloro-2-[(trifluoromethyl)sulfanyl]benzene having structure (1-2): 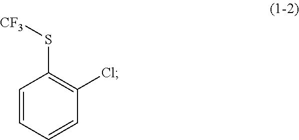 [SMILES: FC(F)(F)Sc1ccccc1Cl]
(b) contacting 1-chloro-2-[(trifluoromethyl)sulfanyl]benzene (1-2) with an oxidizing agent to thereby prepare 1-chloro-2-(trifluoromethylsulfonyl)benzene having structure (1-3): 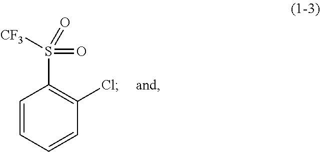 [SMILES: O=S(=O)(c1ccccc1Cl)C(F)(F)F]
(c) reacting 1-chloro-2-(trifluoromethanesulfonyl)benzene (1-3) with chlorosulfonic acid and then with thionyl chloride to thereby prepare 4-chloro-3-(trifluoromethanesulfonyl)benzene-1-sulfonyl chloride having structure (1-4).

21. The method of claim 20, wherein the method is continuous from the preparation of 1-chloro-2-[(trifluoromethyl)sulfanyl]benzene having structure (1-2) from 2-chlorobenzene-1-thiol having structure (1-1) of step (a), and from the preparation of 1-chloro-2-(trifluoromethylsulfonyl)benzene having structure (1-3) from 1-chloro-2-[(trifluoromethyl)sulfanyl]benzene having structure (1-2) of step (b).

22. A compound having structure (1-4):

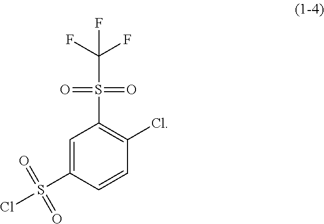 [SMILES: O=S(=O)(Cl)c1ccc(Cl)c(S(=O)(=O)C(F)(F)F)c1]

23. A compound having structure (1-5): 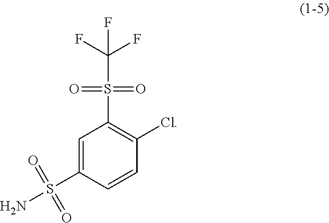 [SMILES: NS(=O)(=O)c1ccc(Cl)c(S(=O)(=O)C(F)(F)F)c1]
